(19) United States
(12) Patent Application Publication
(10) Pub. No.: US 2025/0298982 A1
(43) Pub. Date: Sep. 25, 2025

(54) MACHINE LEARNING FOR AGGREGATING AND EVALUATING DATA FROM A SENSOR ENABLED ENVIRONMENT

(71) Applicant: LogicMark, Inc., Louisville, KY (US)

(72) Inventors: Chia-Lin Simmons, Moraga, CA (US); Rafael Saavedra, Cupertino, CA (US); Peter Williams, Belmont, CA (US)

(21) Appl. No.: 19/089,555

(22) Filed: Mar. 25, 2025

Related U.S. Application Data
(60) Provisional application No. 63/569,575, filed on Mar. 25, 2024.

(51) Int. Cl. G06F 40/30; A61B 5/00

(52) U.S. Cl. CPC G06F 40/30; A61B 5/4088; A61B 5/4803; A61B 5/7267; A61B 5/7282; A61B 5/746

(57) ABSTRACT

Machine learning for aggregating and evaluating data from a sensor enabled environment (SEE) may be provided by receiving training data from at least one SEE related to behavioral, health, wellness, and safety (BHWS) events affecting at least one person under monitoring (PUM); developing a language of wellness (LoW) syntax for a linguistic artificial intelligence or machine learning (AI/ML) model by aligning the BHWS events with a pattern framework indicative of behaviors of the PUM; training the linguistic AI/ML model based on occurrences of the BHWS events in the training data and the LoW syntax such that the linguistic AI/ML model is configured to: generate a predicted BHWS event based on a series of behaviors observed for a particular PUM in a particular SEE; and generate a predictive alert in response to identifying that the predicted BHWS event disobeys the LoW syntax.

### CROSS-REFERENCES TO RELATED APPLICATIONS

The present disclosure claims benefit of U.S. Provisional Patent Application Ser. No. 63/569,575 titled “MACHINE LEARNING FOR AGGREGATING AND EVALUATING DATA FROM A SENSOR ENABLED ENVIRONMENT”, which was filed on Mar. 25, 2024, and is incorporated herein in its entirety.

### BACKGROUND

Sensor-enabled environments may include one or more fixed location sensors, devices or systems installed in an environment or one or more mobile sensors, devices or systems that are present in the environment, all of which can be initialized, calibrated or configured for monitoring the health and wellness of one or more person under monitoring (PUM) in that environment. Data from a sensor-enabled environment may be processed to determine whether one or more health events has occurred or is likely to occur.

### SUMMARY

Systems, methods, and apparatuses are provided for training and implementation of a machine learning model for aggregating and evaluating data from a sensor enabled environment (SEE) for health and wellness care management for one or more persons under monitoring (PUM). In an example, a system comprises a sensor-enabled environment, a memory, and a processing device configured to receive data from the sensor enabled environment, align the data with at least one pattern framework indicative of a behavior of a person under monitoring, evaluate the at least one pattern framework to detect or predict a wellness event, and send an alert indicative of the detected wellness event.

In another example, a method comprises receiving data from the sensor enabled environment, aligning the data with at least one pattern framework indicative of a behavior of a person under monitoring, evaluating the at least one pattern framework to detect or predict a wellness event, and sending an alert indicative of the detected wellness event.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the Figures and the Detailed Description. Moreover, it should be noted that the language used in this specification has been principally selected for readability and instructional purposes, and not to limit the scope of the inventive subject matter.

### BRIEF DESCRIPTION OF THE DRAWINGS

The description will be more fully understood with reference to the following figures, which are presented as exemplary aspects of the disclosure and should not be construed as a complete recitation of the scope of the disclosure, wherein:

FIG. 1 illustrates a block diagram of an example contextual categorization and classification data flow, according to example embodiments of the present disclosure.

FIG. 2 illustrates a block diagram of an example system performing categorization and classification, according to example embodiments of the present disclosure.

FIG. 3 illustrates a block diagram of an example machine learning system, according to example embodiments of the present disclosure.

FIG. 4 illustrates a block diagram of a token management dataflow, according to example embodiments of the present disclosure.

FIG. 5 illustrates a block diagram of a state management dataflow, according to example embodiments of the present disclosure.

FIG. 6 illustrates a block diagram of an attention and focus dataflow, according to example embodiments of the present disclosure.

FIG. 7 illustrates a block diagram of a token management dataflow for responses, according to example embodiments of the present disclosure.

FIG. 8 illustrates a flowchart for an example method for monitoring a SEE, according to example embodiments of the present disclosure.

FIG. 9 is a flowchart of an example method for generating and maintaining a linguistic AI/ML model for machine learning for aggregating and evaluating data from a sensor enabled environment, according to embodiments of the present disclosure.

FIG. 10 is a flowchart of an example method for deploying and using a linguistic AI/ML model for machine learning for aggregating and evaluating data from a sensor enabled environment

FIG. 11 illustrates an example computing device, as may be used as a controller in a SEE to monitor a PUM, as part of a sensor monitoring a PUM, as part of a central or distributed service providing calibration systems for generating and curating AI/ML models for distribution to the SEEs, and the like, according to embodiments of the present disclosure.

### DETAILED DESCRIPTION

Techniques are disclosed herein for training and implementation of a machine learning model for aggregating and evaluating data from a sensor enabled environment (SEE) for health and wellness care management for one or more persons under monitoring (PUM). Monitoring of some individuals may be desirable for the health, wellbeing, and personal safety of those individuals. This is particularly the case for elderly individuals who may have limited memory, for example. Such monitoring, however, introduces significant challenges regarding scalability and quality of life.

Existing techniques for monitoring individuals for health reasons typically require than an individual relocate to a facility equipped with personnel and equipment specialized to perform such monitoring. Besides the obvious loss of freedom that this entails, monitoring for certain conditions can be quite invasive, and patient maltreatment can be a chronic problem for such facilities. One potential solution to this problem is placement of sensors around an individual's living spaces to remotely monitor that individual. This strategy poses additional problems, however. Namely, a single monitored individual may generate exceptionally large quantities of data which can be difficult to monitor to a satisfactory level. Employing large numbers of people to monitor this data would be impractical, especially since much of the monitoring time is uneventful to such a degree that proper attention may be difficult to maintain. It is therefore desirable to implement a system which can automatically aggregate and evaluate data from one or more SEE at scale.

Systems and methods of the present disclosure achieve this aim by training a machine learning model to recognize a variety of patterns in data that may be produced by a sensor-enabled environment. This machine learning model may align this data with one or more corresponding pattern frameworks, which may then be used to determine one or more corresponding behaviors of a person under monitoring (PUM). Once the one or more behaviors have been determined, the machine learning model may correlate the one or more behaviors with likely health or wellness events which are occurring or which may soon occur to the PUM. The machine learning model may send an alert indicative of the health or wellness event to monitoring personnel, thereby potentially limiting human involvement to one or more PUM who may be in need of additional attention or intervention.

A SEE may generate one or more data sets from one or more sensors, devices, or systems deployed or present within the SEE. These data sets may be arranged into one or more patterns. These pattern arrangements may, in whole or in part, be undertaken at an “edge”. For example, these pattern arrangements may be determined by the one or more sensors, devices, or systems or by a receiving system, for example a hub, in any arrangement. In some example embodiments, these patterns may be formulated in pattern frameworks based at least in part on behaviors of one or more PUM as they undertake their daily activities or routines. These patterns may in turn form contextual behaviors that can, at least in part, represent a PUM that is domiciled in such a SEE as they engage in their daily activities and life.

Each of these patterns may be specified in differing arrangements. For example, they may be arranged according to one or more categorizations including but not limited to an ontology, taxonomy, or other organization. These categorizations may be based on a number of factors, and in some embodiments may be generated by one or more artificial intelligence or machine learning (AI/ML) modules. In some embodiments, these categorizations may represent the behaviors of a PUM in a SEE and may be considered or evaluated in and across multiple categorizations including, for example, spatial, temporal, contextual, health, or wellness.

In some embodiments, a pattern may span more than one of these categorizations or may contribute to or comprise another one or more categories or categorizations, for example those generated through the use of one or more AI/ML modules using one or more training data sets including, for example, the data sets or patterns generated by one or more SEE.

Using these example categorizations, a PUM's behaviors may be represented in the form of spatial (e.g., locational), temporal (e.g., in or over a time period), contextual behavior (e.g., the current activity of the PUM), or health and wellness (e.g., including health and wellness monitoring). This example categorization comprising patterns may include the data sets generated by, at least in part, the one or more sensors, devices, or systems deployed or present in a SEE where the PUM or other stakeholders are under monitoring.

The relationships between the categories, the dimensions, or feature sets thereof may be deterministic. An example of this may be sensors that are located in specific areas may form part of a spatial group or category, such as a bedroom or similar. The relationships between the categories, the dimensions, or feature sets thereof may also be non-deterministic. An example of this may be a set of sensors that generate data based on haptics, such as foot fall and the like.

Each of these categories may have one or more dimensions or one or more feature sets which may comprise such data sets or the patterns in any arrangement. In some embodiments, one or more topological representations may be used to manage such dimensions or feature sets including, for example, in one or more repositories of patterns, dimensions, or features.

This holistic representation of a SEE and the one or more PUM domiciled therein enables a comprehensive monitoring approach that, based on the various data sets or patterns, may be calibrated and optimized to identify variations in the context and behaviors of a PUM that may have a health and wellness impact across various time periods, in various locations, or in the context of their overall wellness and health.

One of the aspects of this approach may be the use of AI/ML in combination with digital twins and including in some embodiments one or more physics engines such that variations in the overall PUM wellness and health may be identified in a detailed responsive ongoing or timely manner. This approach enables and supports a more proactive approach to the identification, detection, mitigation, or alleviation of the health and wellness issues that challenge us all as we age.

The use of this combination of SEE, AI/ML, digital twins or physics engines in combination may provide significant benefits for the PUM, whilst respecting their privacy, quality of life and their inherent life choices.

The categories described herein may be evaluated in isolation or in any combination to create differing perspectives on the PUM overall health conditions. For example, if a PUM has limited movement, such as issues with their mobility, the care hub systems may be calibrated to account for an uneven footfall of that PUM and in consequence the dimensions of the spatial categorizations, for example, may be adjusted accordingly.

An aspect of this approach may be identification of structures comprising patterns, categorizations, dimensions, or features represented, for example, as multi-dimensional manifolds in an efficient manner, where an outline of the structure is evident with minimal possible data sets. For example, the use of convolutional neural networks in combination with recurrent neural networks may create a minimal structure based on these data sets. This may include the use, for example, of large language models (LLM).

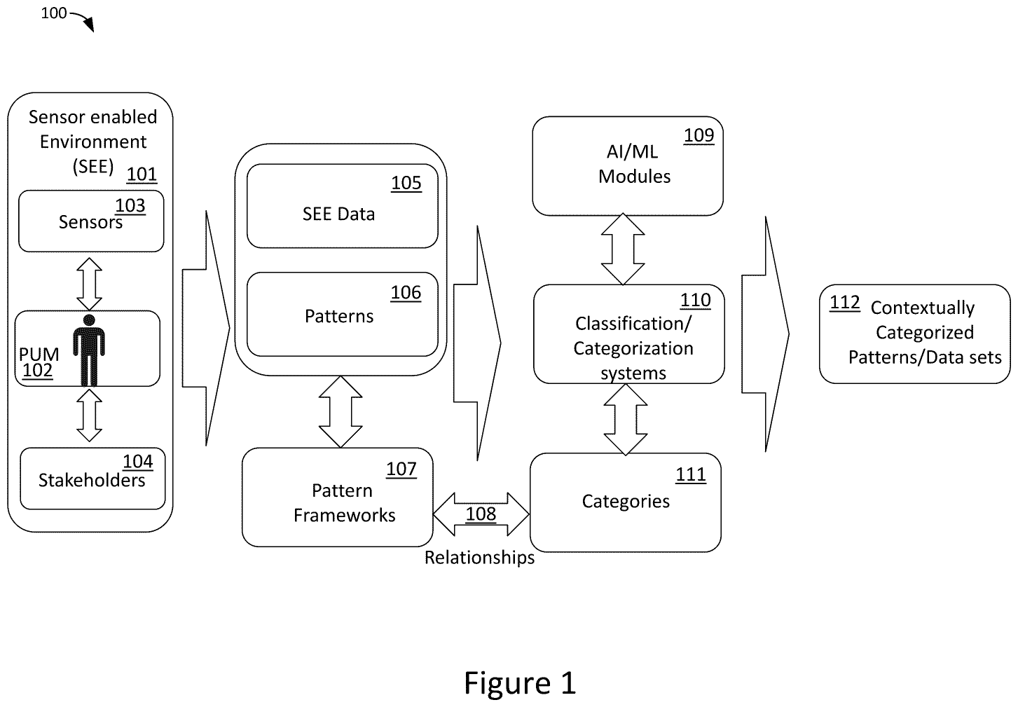

FIG. 1 illustrates a block diagram of an example contextual categorization and classification data flow 100, according to example embodiments of the present disclosure. In this example, a SEE 101 includes sensors, devices, or systems 103 for monitoring a PUM 102 or stakeholders 104. These sensors 103 generate data sets 105 representing SEE 101, PUM 102, and Stakeholder 104 activities. These data sets 105 may be aligned to pattern frameworks 107 to generate patterns 106 comprising, at least in part, SEE data 105 and Pattern Frameworks 107. The SEE data 105 or patterns 106 may be communicated to one or more classification/categorization systems 110. These systems 110 may use one or more categories 111 or one or more AI/ML modules 109 to align SEE data 105 and patterns 106 to generate contextually categorized patterns or data sets 112. In some embodiments, relationships 108 between, for example, pattern frameworks 107 and categories 111 may be persisted in one or more repositories such as in a graph database.

In some embodiments, a set of categorizations may be used to configure the patterns of data generated by the SEE into actionable arrangements that may be employed for managing the health and wellbeing of the PUM. This categorization may provide the basis for one or more techniques for algorithmic processing of these categories and the patterns or data sets they include so as to create one or more responses, including but not limited to response candidates, that may be deployed.

The patterns or data sets and their formulation into categories may involve the use of one or more AI/ML systems where, for example, these patterns or data sets may form one or more training sets for such AI/ML modules.

Several of these initial example categorizations are outlined herein, however there may be additional categorizations that are generated by the one or more AI/ML modules or created by initialization, calibration, or configuration of the one or more sensors, devices, or systems of the SEE or the one or more hub systems in any arrangement.

A spatial category may be location centric, for example a room in an environment, a portion of a room, a space around a room, or encompassing a feature of a room such as a sofa, or a functional area, such as a food preparation area. The spatial category may include one or more volumetric metrics based on a location, where boundary of the spatial domain may be determined, at least in part, by the boundaries of the space, such as the dimensions of a room.

In some embodiments, there may be a spatial category that is centered on the PUM such as, for example, a spatial domain with a diameter minimum of “an arms length”, representing a reach of the PUM or a “legs length” representing their stride, both of which may be related to PUM specifications such as a height, weight, gait, leg/arm dimensions, or other metrics. For example, this may include a typical 1 to 1.5 Meter circular diameter based on a body center line and having a height based upon the PUM's height plus, for example, 1 meter.

In this manner the spatial categorization of the PUM may be evaluated in relation to other spatial aspects of the environment, for example to ascertain when the PUM may interact with another spatial entity. This may be particularly useful in determining potential impacts for a PUM when navigating an environment.

Temporal categorizations may include 24 hour clock time or time periods related to the one or more behaviors of the PUM, including those of the one or more contextual behaviors of the PUM. The 24 hour clock time may be used to segment the activities of a PUM into, for example, sleeping, exercising, eating, and other activities.

Contextual behaviors may include one or more pattern frameworks that have, for example, been deployed as part of a calibration of a SEE. These contextual behaviors may include one or more data sets generated by the one or more sensors, devices, or systems which are aligned with one or more pattern frameworks to create a contextual behavior that is specific to a PUM. This approach may also be applied to one or more other stakeholders with whom the PUM interacts, for example a carer who may undertake food preparation for the PUM.

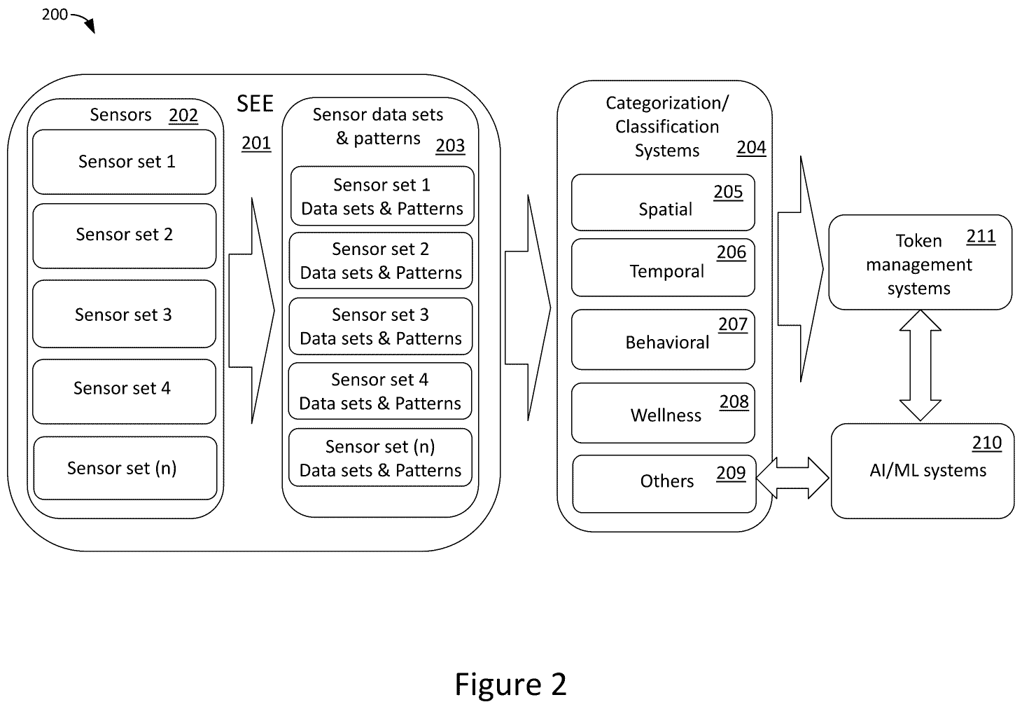

FIG. 2 illustrates a block diagram of an example system 200 performing categorization and classification, according to example embodiments of the present disclosure. A SEE 201 may comprise a set of sensors 202 in any arrangement, which may generate sensor data sets or patterns 203. These sensors 202, data, sets or patterns 203 may be communicated to one or more categorization/classification systems 204 where, for example, one or more categorization/classification may be undertaken. Each of these may include data about the one or more sensor sets 202, the data sets, or patterns they generate 203 and their relationships to the SEE 201 in any arrangement. For example, categorization/classification systems may include spatial categories 205, temporal categories 206, behavioral categories 207, wellness categories 208, or other categories 209, which for example may be identified by one or more AI/ML systems 210. These categorizations or classifications may be communicated to one or more token management systems 211, where they may be formed into one or more tokenized representations.

The health and wellness categorizations may comprise, at least in part, a healthcare profile (HCP) of the PUM insofar as the HCP specifies health and wellness reasons for monitoring. The health and wellness categorizations may include, for example one or more health and wellness events, such as those detrimental to a PUM (e.g. a fall) where the data sets or patterns generated by the one or more sensors, devices, or systems of a SEE are arranged in one or more pattern frameworks representing such an event. These event pattern frameworks may include, for example, falls, breathing difficulties, mobility difficulties, heart or other organ difficulties, injuries (such as cutting with a knife when preparing food), or other events. In some embodiments these event pattern frameworks may be prioritized based, at least in part, on the specific of the reasons for the monitoring, represented by the HCP. In this manner, such operations as attention or focus processing as described herein may be prioritized to those most likely event frameworks.

These categorizations may comprise data sets, patterns, features, or dimensions that are specific to that categorization. For example, a temporal category may include a timeline dimension representing previous, current, and future time periods.

In some embodiments, there may be dimensions or feature sets that span one or more categorizations, such as those that involve spatial, temporal, or behavioral elements. For example, this may include quality of life dimensions such as those associated with diet, exercise, or hobbies.

In some embodiments there may be further categorizations that are formulated, at least in part, by the one or more AI/ML modules. This may include a determination by such modules of feature sets, dimensions, or other characteristics of spatial, temporal, behavioral, or wellness categories as well as further categories that may be generated by the AI/ML modules.

In some embodiments, these further categories may be evaluated, for example, in one or more digital twins in collaboration in some circumstances with one or more physics engines or one or more sets of specifications, possibly including the HCP, to determine, at least in part, an accuracy, reliability, or utility of the specifications. This can in some embodiments include evaluation by one or more human actors, including the PUM or other stakeholders.

In some embodiments a framework, such as those that form, at least in part, one or more categorizations, may involve one or more digital twins which may be initialized with data sets or patterns derived from one or more calibrated sensors or repository-based data sets from similar situations. These digital twins may have an associated set of strategies that represent behaviors of PUM from this initial state. These may be aligned with game theory, vectors, topologies, or other representations. These strategies may represent the most likely behavior variations, based at least in part on the SEE data sets, AI/ML modules, game theory strategies, or physics engines.

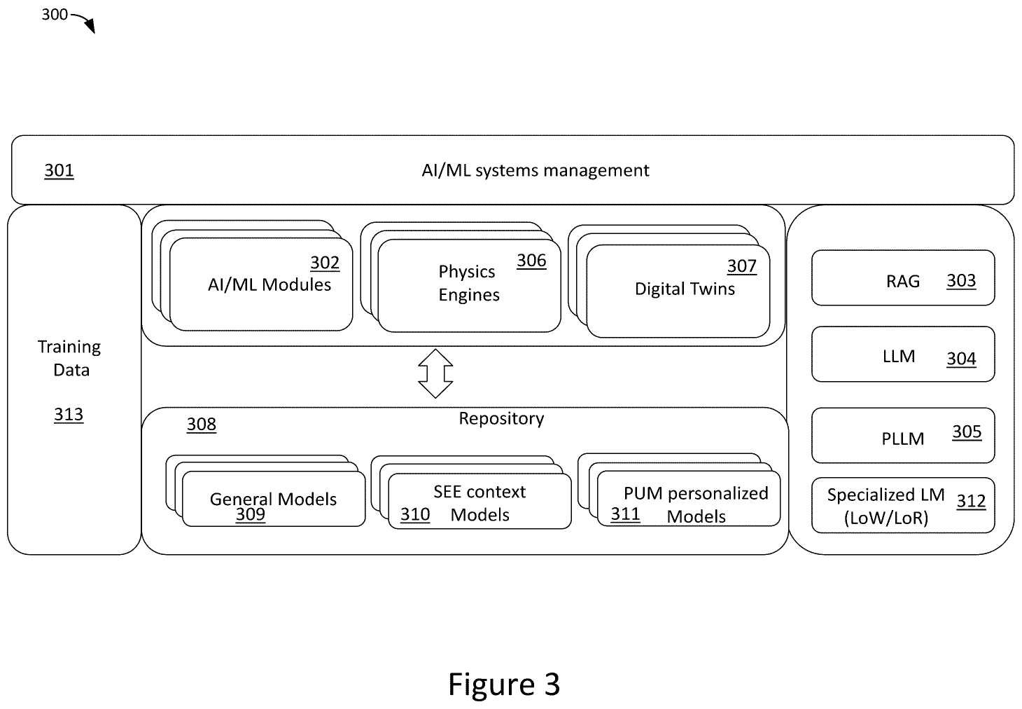

FIG. 3 illustrates a block diagram of an example machine learning system 300, according to example embodiments of the present disclosure. In this example an AI/ML management system 301 comprises or manages one or more AI/ML modules 302, one or more physics engines 306, or one or more digital twins 307 in any arrangement. These AI modules 302, physics engines 306 or digital twins 307 may generate, based on one or more sets of training data 313 including, for example, sensor data sets 105, patterns 106, or categorizations/classifications 112, some of which may be represented by one or more tokens, to create one or models held in one or more repositories 308. For example, such models may include general models 309, SEE context models 310, or PUM personalized models 311. In some embodiments one or more AI/ML systems may be employed, such as retrieval augmented generation (RAG) AI 303, large language model (LLM) AI 304, private large language model (PLLM) AI 305, or one or more specialized language model (LM), such as one or more care language (Language of wellness/language of care response) 312.

In some embodiments, there may be one or more languages representing care of one or more PUM in one or more SEE. These languages may be used for communication between one or more sensors, devices, or systems, including but not limited to care hubs or care processing systems, and may, in whole or in part, support communications between machines or humans in any arrangement.

Such languages may form specialized representations of context, events, actions, situations, or other characteristics of one or more PUM domiciled or present in one or more SEE. In some embodiments these languages may comprise sets of tokens representing various care conditions and states thereof, and may include spatial, temporal, behavioral, or wellness categorizations of the one or more data sets or patterns generated by the one or more sensors, devices, or systems of one or more SEE. For example, such categorizations may include relationships between and amongst the one or more sensors, devices, or systems, including care hubs and care processing systems, and the data sets or patterns they generate.

In some embodiments one or more token management systems may be employed for the generation of one or more tokens representing, for example, sensors, devices, or systems or the data sets or patterns they generate. In the same manner, pattern frameworks, categorizations, classifications, or relationships between these entities may also be formed into one or more tokens by one or more token management systems. The token management systems may employ one or more cryptographic techniques that may be applied to one or more tokens in support of one or more security, privacy, or distribution schemas which can, for example, form part of one or more care hub or care processing systems.

These tokenized representations may, for example, in whole or in part, be aligned with one or more large language models, including private or specialized language models. For example, a carer may be able to, using one or more care languages, query a wellness state of a PUM.

In some embodiments, for example, a care language may be instantiated as a language of wellness (LoW), and may be employed by a sensor, device, or system communication means for one or more machines, including but not limited to other sensors, devices, or systems, including, for example, care hubs or care processing systems with, at least in part, a purpose of monitoring a wellness and health of a person under care in a sensor enabled environment.

The one or more data sets or patterns that may be generated by the one or more sensors, devices, or systems embedded or present in a SEE may, in some embodiments, be represented in the form of a language of wellness (LoW). This language, in common with other languages, may have a set of expressions, a syntax, and a set of semantics. For example, the expressions may comprise representations of one or more data sets or patterns that are tokenized versions of those expressions. In some embodiments, tokens representing data sets or patterns from one or more sensors, devices, or systems may be used in combination to form one or more further tokens representing one or more patterns.

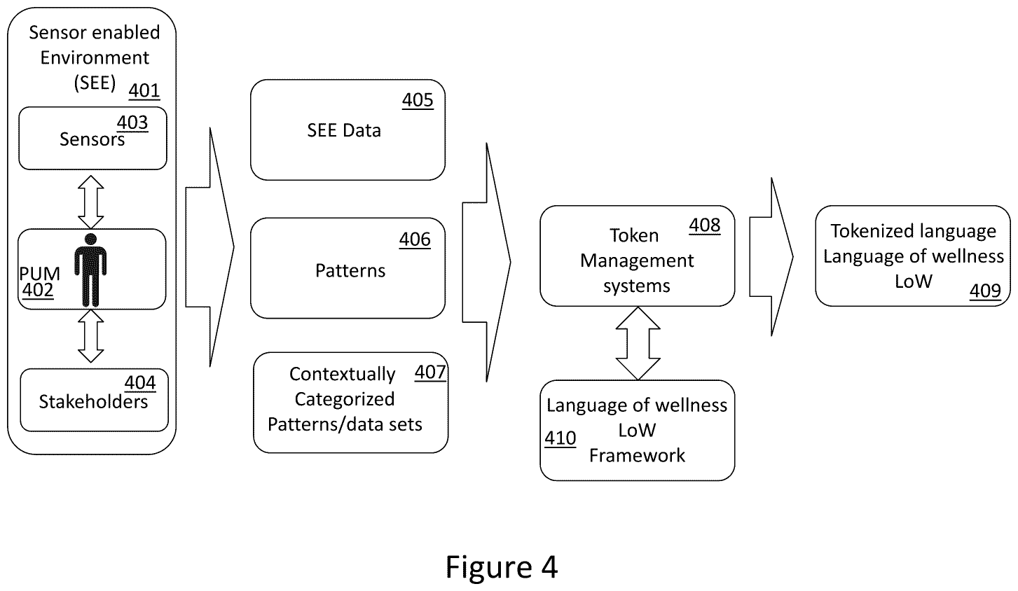

FIG. 4 illustrates a block diagram of a token management dataflow 400, according to example embodiments of the present disclosure. For example, a SEE 401 may include one or more PUM 402 or one or more stakeholders 404 which are monitored by one or more sensors, devices, or systems 403. The data sets 405, patterns 406, or contextually categorized patterns or data sets 407 generated by such SEE 402 may be communicated to one or more token management systems 408 to generate, for example using a language framework 410, one or more tokenized care languages, for example a language of wellness (LoW) 409.

For example, a pattern framework may comprise a set of tokens which may be open or closed. In some embodiments, a pattern may comprise, for example, token N out of a set of Y tokens, where N is a subset of Y. In this example, a pattern token (N) may be specified as an algorithm where certain tokens representing data sets from the one or more sensors, devices, or systems in a SEE are arranged in a specific combination.

The tokenized patterns or data sets may form the language of wellness (LoW), whereby the language may include tokens that are created, at least in part, by one or more ML/AI systems and may form part of a model developed by such an AI/ML module that is, in part or in whole, trained on the data sets or patterns of the one or more sensors, devices, or systems of one or more SEE.

Tokenization enables processing of data sets into arrangements representing combinations. Some examples of this may include but are not limited to motion detection, haptic footfall detection, audio detection, or other detections which may be combined to represent, for example, a PUM moving from one location in an environment to another. In this example the token or set thereof may be part of a spatial categorization (e.g. moving from spatial location A to spatial location B), a temporal categorization (e.g. at time T for duration D), a contextual behavior (e.g. moving from a couch corresponding to spatial location A to a kitchen area corresponding to spatial location B) for morning coffee, or a wellness and health categorization (e.g. footfall and audio sensor data sets outside parameter thresholds, potentially indicating the PUM having mobility difficulty).

Tokenization may be employed where, for example, a token may include a set of segments comprising one or more data sets from one or more sensors, devices, or systems or other sources including other tokens in any arrangement. Tokens may also include one or more thresholds or one or more relationships with one or more patterns. These token segments may be available to one or more sensors, devices, systems, care hub services, or care processing services, for example using one or more cryptographic key regimes or other access control paradigms.

In some embodiments a monitoring language may include one or more known health and wellness events, including those that are detrimental or beneficial to a PUM, which may be represented by patterns, data sets, or tokens.

A language comprising a set of tokens may in some embodiments be processed by a Large Language Model where the tokens, forming such language, are processed by the LLM such that a context of any one token is constrained by the model or capabilities of the LLM, such that this context is, at least in part, determined by such processing.

In some embodiments, the language of wellness (LoW) may include one or more categorizations which may represent a syntax of the language. These categorizations may include, for example, spatial, temporal, behavioral, or health and wellness. This syntax may be extensible where, for example, one or more AI/ML systems may generate, based on one or models created from one or more training data sets comprising the data sets or patterns of the one or more sensors, devices, or systems of one or more SEE, further categorizations or classifications that are represented as tokens and form part of the syntax of the language.

In some embodiments a relationship between categories may be determined by one or more care hubs or care processing systems such that a set of categories (e.g. spatial, temporal, behavioral, or health and wellness) are given priority through, for example, specification, declaration, weighting, or similar over further categories that are generated by the one or more AI/ML modules. In some embodiments one or more physics engines may be employed to evaluate the one or more categories generated by the one or more AI/ML modules to ensure, at least in part, that the categories comply with applicable physics of the configured physics engine.

In some embodiments, there may be ontologies or taxonomies of these categories, as well as other organizational arrangements.

In some embodiments certain sensors, devices, or systems may represent data sets or patterns thereof in specific tokens. For example, a time measuring sensor may generate temporal tokens or a haptic sensor may generate haptic tokens. However, these tokens may be combined and synchronized to reflect sensor capabilities that may have common characteristics. In some embodiments, this may include but is not limited to identity, time, or location.

In some embodiments a care hub or care processing system may be configured such that specific sensors, devices, or systems have specified relationships with one or more categories. This may include specifying one or more sensors, devices, or systems as providers of tokens representing the data sets or patterns generated by such one or more sensors, devices, or systems as forming part of a particular spatial, temporal, behavioral, wellness, or other categorization or classification that can, at least in part, form part of a language of wellness (LoW).

In some embodiments, the raw data sets generated by the one or more sensors, devices, or systems may form training data sets for one or more AI/ML modules. This may be the case when an AI/ML module is tasked with generating models that are independent of any foreknowledge, such as pre-existing, specified, or deterministic categorizations or classifications. In this example, unsupervised learning techniques may be applied.

In some embodiments, state, including context represented by one or more data sets, patterns, categorizations, or classifications, may be tokenized and form part of the LoW. For example, in some embodiments, there may be certain elements of LoW that represent state or context such as, for example, temporal elements including past, current (including time periods), future, spatial elements, such as locational, one or more contextual behaviors derived, at least in part, form one or more pattern frameworks, wellness and health elements, for example those based, at least in part, on event frameworks.

In some embodiments, a LoW may enable differing perspectives using AI/ML models that represent these differing perspectives. An example of these differing perspectives may be those of differing stakeholders who interact with a PUM.

In some embodiments a private large language model (PLLM) may be employed to generate one or more models representing behaviors or interactions of a PUM in a SEE. For example, the PLLM may use the data sets or patterns generated by the one or more sensors, devices, or systems of a SEE to generate, at least in part, one or more models representing the PUM in a SEE, for example using a language of wellness (LoW).

In some embodiments the relationship between PUM behaviors represented, for example, by one or more contextual behavior and state may, at least in part, be determined by a language of wellness (LoW).

Such a language, comprising a tokenized representation of the state of the PUM in a SEE, may be aligned with one or more of these states. In some embodiments such sates may be quantized such that a specific set of data generated by the one or more sensors, devices or systems employed or present in the SEE, represented as patterns, for example, may form one or more quantized states.

In some embodiments, this quantization of state may be based, at least in part, on one or more categorizations comprising a categorization and one or more variables expressed in some embodiments as metrics, which may be employed as weightings representing these variables and where state may be a specific balance between each of the variables in the form of a ratio forming a topological representation of one or more dimensions such that state may be represented in some embodiments as an enclosed space within a topology. Each of these spaces may have a shape and may vary depending on the data sets, patterns, or weight variables of dimensions that form boundaries of the shape.

State shapes may be formalized as a set of classified entities including, for example, quiescent states where a state shape may change based, at least in part, on the data sets or patterns of the sensors, devices, or systems forming the SEE. In some embodiments, these changes may be expressed as vectors, which may include a rate of change (velocity), and a rate of rate of change (acceleration).

Each of these vectors may be used, at least in part, by one or more AI/ML systems to predict likely state shapes which represent the data sets or patterns that in turn represent PUM behaviors. For example, each state shape may have a finite set of potential shape shifts where the vectors represent relationships between these shapes.

In some embodiments one or more AI/ML modules may be employed in combination with one or more Digital Twins to generate one or more models representing potential shape shifts, one or more state, state changes, state transitions, or other state characteristics of the PUM domiciled in a SEE.

In some embodiments these shapes may be compared and evaluated from multiple PUM, where the identity of the PUM is not discernable from the state shape while the state shape remains effective to, at least in part, calculate likely vectors based on aggregated set of shapes representing a diaspora of PUM with similar HCP.

For example, a dimension may be expressed as a vector from a point which may be an origination or an intersection of one or more other dimensions. In this manner a vector of each dimension may have a differing trajectory such that an angle between the vectors may represent a degree of alignment of the dimensions. For example, if the angle between the dimension and the vector is acute, then the alignment of the dimensions may be evaluated to determine, at least in part, one or more metrics that may represent a quality of life (QoL) of the PUM in their SEE domicile as these dimensions form part of the representation of the state of that PUM in their SEE.

A set of metrics may be employed by one or more AI/ML models as representations of QoL of a PUM, and may be used, at least in part, as an element of an improvement strategy. This may be expressed, for example, in the form of game theory strategies where payoffs include metrics representing the quality of life of the PUM.

These strategies may include predictive and deterministic elements where, for example, the deterministic elements are based, at least in part, on the data set representations from the SEE of the domiciled PUM. The predictive elements may be based, at least in part, on game strategies that include state shapes where the associated vectors represent potential changes to those states quantized in the form of game theory strategies. In some embodiments this may include the deployment of AI/ML modules and one or more physics engines to, at least in part, ensure that the predicted vectors are within characteristics and capabilities of the PUM in their SEE enabled domicile.

In some embodiments, temporal dimensions may have vectors that may be forward (predictive) looking from a current time or rear looking (historical). In either case, a vector perpendicular to a temporal dimension may be indicative of multiple parallel dimensions within the state space.

In some embodiments, the state of one or more categorizations including but not limited to contextual behaviors, spatial, temporal, or wellness and health may be evaluated on a continuous or sampled basis to determine, at least in part, an earliest indication of a health or wellness event affecting the PUM. This evaluation can, in some embodiments, include one or more verifications that the early indications, represented by a change in state, are confirmed by multiple sensors, devices, or systems in a manner configured to reduce any false positives.

These combinations of patterns, data sets, or tokens may be evaluated to ascertain that the change in state conforms to one or more known patterns, data sets, or token relationships which, in whole or in part, may represent a health and wellness event, for example a fall.

In some embodiments, one or more topologies may be employed, in combination with one or more digital twins, to, at least in part, generate one or more predictive representations of the one or more states of the SEE, including the PUM therein.

For example, state shapes comprising patterns arranged into spatial, temporal, behavioral, or wellness based categorized data sets may be used in one or more digital twins where one or more characteristics of these patterns may be varied to, at least in part, determine thresholds for each dimension (spatial/temporal/behavioral/wellness), including the relationships between them.

In some embodiments, one or more AI/ML modules may be used to modulate the digital twin data sets that are derived, at least in part, from the SEE data sets. These modulations may be within boundaries that are representations of the possible data sets that one or more sensors, devices, or systems may generate in a SEE. The modulations may be in multiple dimensions and may involve correlated feature sets, that is to say if one feature is prominent in a particular modulation, then another may degenerated in response. The modulations may be temporal or may be based, at least in part, on contextual behaviors of a PUM, including health and wellness events. For example, a modulation may be based on a recuring PUM wellness behavior, for example a cough, which may be aligned to a seasonal occurrence (e.g. an allergy).

For example, if pattern A data set A1 becomes data set A2, which for example may be determined, at least in part by such data exceeding one or more thresholds, this may indicate a change in state. Such state change can, for example, be communicated to one or more AI/ML modules for evaluation including but not limited to extrapolation, for example using one or more digital twins to generate one or more sets of possible new states. These new states may be validated by one or more physics engines or one or more sets of specifications (e.g. HCP). These potential states can, for example, be compared or validated against other known patterns, such as those stored in one or more repository, or are identified as new patterns or states. These new patterns or states may include health and wellness events.

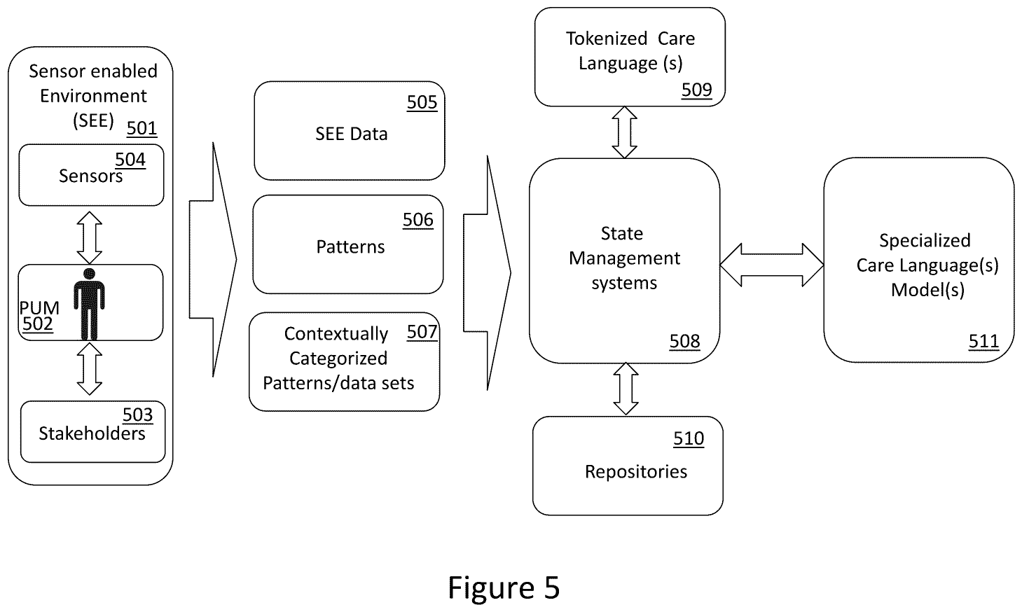

FIG. 5 illustrates a block diagram of a state management dataflow 500, according to example embodiments of the present disclosure. A SEE 501 that includes one or more PUM 502 or one or more stakeholders 503 monitored by one or more sensors, devices, or systems 504 may generate SEE data 505, patterns 506, or contextually categorized/classified patterns/data sets 507 in any arrangement which may be communicated to one or more state management systems 508. The state management systems may interact with one or more tokenized care languages 509, repositories 510, or specialized care language models 511 to create, at least in part, a representation of the state of the SEE 501 or elements thereof, including one or more PUM 502, stakeholders 503, sensors 504, SEE data 505, patterns 506, or contextually categorized/classified patterns/data sets 507 including, for example, in the form of one or more tokenized care languages 509.

In some embodiments, input datasets may be processed and interpreted more accurately, faster, more effectively, and with more efficient use of resources if contextual information is applied and taken into account along with input datasets. Context may play a crucial role in interpreting and responding to inputs as context may allow a system to consider surrounding circumstances, previous inputs, and relevant information when interpreting inputs or identifying patterns.

Contextual information may include one or more data sets, patterns, pattern frameworks, event frameworks or other frameworks, specifications, calibrations or configurations, circumstances, history, or other relevant information about the PUM, the SEE, stakeholders such as caregivers, or external elements, such as weather, local events, legal context, or other factors. For example, during heat waves, a system may interpret sensor input that indicates behavioral changes in the PUM, such as fewer hours of sleep or slightly higher blood pressure as an increased risk of a heat-related critical health event.

One or more mechanisms may be used and combined in order to consider context when processing input datasets. In some embodiments, context management mechanisms may be available with AI/ML systems, where various techniques may be used to capture, retain, and use contextual information across different modalities enabling more nuanced and accurate responses to inputs. Mechanisms such as transformers, which use self-attention mechanisms to assign different weights to input tokens, allow a model to consider relevant context, including past input datasets.

In some other embodiments, context management may be done by using domain-specific training, where models are pre-trained on large datasets to learn general features and context, then fine-tuned with additional training using domain-specific datasets or through mechanisms such as transfer learning. This may result in models that are suited to specific domains or contexts. Combinations of these specialized models may be selected, based on their specific contexts, to interpret or process inputs.

In some embodiments, inputs and contextual information may be used to select a most appropriate module to interpret or process the inputs. This selection may be accomplished by various mechanisms, such as using ML-based classifiers or applying maps, rules, patterns, or pre-defined flows or relationships. This may be applied at one or more levels and may be performed on raw data sets coming from sensors, devices, or systems, events or patterns coming from pre-interpretation of such raw data allowing for context-based processing at multiple levels and locations within one or more SEE.

The use of categorizations of the one or more data sets generated by the one or more sensors, devices, or systems of a SEE may enable the data sets origins of those data sets to have a relationship with these categorizations. For example, a sensor data set may form part of a temporal category and a spatial category. In some embodiments, each of these sensors, devices, or systems data sets may contribute to a categorization where that contribution may include the data from the sensor, an identity of the sensor, or one or more weightings of that data. These data sets may form one or more patterns which in turn form, in part or in whole, one or more categories. For example, this weighting may be, in part, determined by a relative importance or value of a contribution to a category. This importance or value may, at least in part, be determined by the pattern of which such a data set is a part.

In some embodiments, each of the categorizations may be evaluated individually or collectively and as such one or more weightings may be calculated, bound, or assigned to each of these categories.

In some embodiments, there may be default values for weightings of the one or more patterns or combinations thereof. For example, a pattern forming part of a mobility event which is categorized as a spatial categorization may have a high weighting value as the PUM movement may be, for example, tracked by a set of sensors including but not limited to visual, audio, haptic, radar, or other sensors capable of tracking movement such that the pattern has a high internal consistency. Each of the sensor data sets in aggregate may be determined, for example by one or more pattern recognition systems such as a care hub, care processing system, or an edge processing system in any arrangement, to be consistent with the movement of the PUM from one location to another in a SEE.

In some embodiments, there may be default values for the one or more data sets or patterns that form a pattern framework where, for example, a mobility pattern framework has a set of N patterns which in this example is a closed set, and each of the data sets or patterns in this set has a default value, representing a relative priority of these data sets or patterns that from such a pattern framework.

In some embodiments, an action or event of a PUM may be represented by relative values of the one or more categorizations of the data sets or patterns generated by the one or more sensors, devices, or systems of a SEE. For example, an event may include spatial, temporal, behavioral, or health and wellness categorizations where the one or more data sets or patterns generated by the one or more sensors, devices, or systems form part of these categorizations with differing priority, weightings, or other relative values. Some of these relationships may have default values that are part of the pattern frameworks or may have specified weightings for the one or more categories that they are part of.

In some embodiments, the determination of these weightings may, at least in part, be undertaken by one or more AI/ML modules, where training data for these modules is based, in part or in whole, on the data sets of the one or more sensors, devices, or systems of one or more SEE in which one or more PUM is domiciled.

One aspect of the employment of AI/ML modules may be evaluation or configuration of one or more relationships between and amongst the one or more sensors, devices, or systems, the data sets or patterns generated thereby, the one or more categorizations or classifications, or the events, actions, or other activities of one or more PUM in one or more SEE.

In some embodiments, one or more repository may be used for management or storage of the one or more relationships between the one or more sensors, devices, or systems present in a SEE, the data sets or patterns they generate, or the one or more categorizations or classifications to which they have one or more relationships. In some embodiments such repositories may be in the form of one or more graph databases.

In some embodiments one or more AI/ML modules may be employed to evaluate, manage, classify, identity, or in other manners operate upon a repository and the data stored therein. This may include, for example, use of fuzzy logic and other similar techniques to identify or classify relationships between the elements stored therein. This may include identification or classification of relationships that have not been previously instantiated.

Care hubs or care processing systems may employ one or more AI/ML systems to, in part or in whole, identify a composition of elements in one or more relationships between, for example, one or more sensors, devices, or systems, data sets or patterns generated thereby, classification and categorizations of these data sets, patterns or pattern frameworks in any arrangement, one or more SEE, or one or more PUM.

In some embodiments these relationships may be static or dynamic. For example, a dynamic relationship may be between a worn or carried device (e.g. a pendant or smart phone of a PUM) that is present in a SEE, and the one or more sensors, devices, or systems that are embedded at fixed locations within a SEE.

In some embodiments, evaluation of data sets, patterns, categorizations, or elements thereof may include the use of generative adversarial networks (GAN) networks to, at least in part, determine an optimized balance between the categorizations, patterns, or elements thereof. Generally, a GAN uses two neural networks in an adversarial manner in a zero-sum game, however in addition to this approach there may be two or more neural networks which are configured to act as players in game theory-based games.

In some embodiments relationships between one or more sensors, devices, or systems and the data sets or patterns generated by them may be evaluated, for example, by one or more care hubs or care processing systems to establish dependencies of such relationships. This may be used, in some embodiments, to determine a validity or accuracy of such data sets or patterns to detect any faults with the sensors, devices, or systems.

In some embodiments, one or more sensors, devices, or systems including, for example, care hubs or care processing systems, may have available resources, including but not limited to processing power, storage, communication bandwidth, or other resources. These resources may generally be understood to form constraints on an operating performance of any technology and, in the case of AI/ML operations, may include a time, heat, processing power, training data set, or other factors.

The sensors, devices, or systems deployed or present in a SEE that are employed for the monitoring of a PUM may all have some degree of constraint, such as batteries or power sources, communication capabilities including bandwidth, processing capability, memory or other storage, or other constraining resources.

In situations where resources have limitations, one or more AI/ML model may be used identify which data sets or patterns represent, at a current or predicted time, a most likely resource to represent a health or wellness impact on a PUM. This determination can, in some embodiments, be used to direct those sensors, devices, or systems to generate data sets or patterns that involve the use of those resources of those sensors, devices, or systems to consume those resources to produce data sets or patterns of sufficient fidelity, granularity, or timeliness that may be, at least in part, used to monitor or predict a health or wellness impact on a PUM. This use of the one or more AI/ML modules or one or more care hubs or care processing systems may enable the SEE, in whole or in part, to focus on one or more PUM activities, including behaviors, and may, in some embodiments, be controlled by such AI/ML modules, care hubs, care processing systems, or other configured systems, possibly with human intervention, to focus the attention of such SEE.

In some embodiments, an attention dashboard feature may be presented to one or more humans monitoring a PUM in a SEE. This may include providing a supervisory capability to one or more health professionals, for example, when a fall or other detrimental effect is detected. In some embodiments, such attention management may involve communication of this attention or focus to one or more human, machine, or combination in any arrangement.

One aspect of this approach may be use of, for example, pattern edge detections, including those with static or dynamic thresholds where, for example, one or more systems, including those employing one or more AI/ML modules, may be configured to identify a rate of change, step functions, vectors, topological features, or other metrics that indicate, at least in part, transitions from one state to another.

In some embodiments, a care hub or care processing system may include an attention/focus module that in turn may include one or more AI/ML modules that operate on the one or more data sets or patterns generated by the one or more sensors, devices, or systems of a SEE. This may include the use of one or more digital twins to evaluate or predict the operation of such attention/focus systems, for example, to evaluate an impact on the sensors, devices, or systems and their use including but not limited to consumption of resources.

In some embodiments one or more metrics for attention or focus may be employed to, at least in part, represent a state of the sensors, devices, or systems deployed or present in a SEE.

Context management may also be employed as part of an attention or focus process such that the data sets and or patterns generated by the one or more sensors, devices, or systems of a SEE include contextual information such as relationships of the PUM to other stakeholders, the PUM's contextual behaviors, including patterns thereof, or other relationships that a PUM may have.

For example, an attention or focus process may be initiated based on differences in one or more patterns that may form part of one or more categorizations such as spatial, temporal, behavioral, or wellness and health, where these pattern differences may include, at least in part, contextual data on, about, or involving a PUM. In some embodiments, such contextual data may be employed to augment input data sets, for example those generated by the one or more sensors, devices, or systems of a SEE.

In some embodiments, one or more AI/ML modules, physics engines, or digital twins may be employed to, at least in part, determine which data sets, patterns, or behaviors are prioritized for various levels of attention or focus. This may include the use of digital twins to evaluate a potential for such attention and or focus.

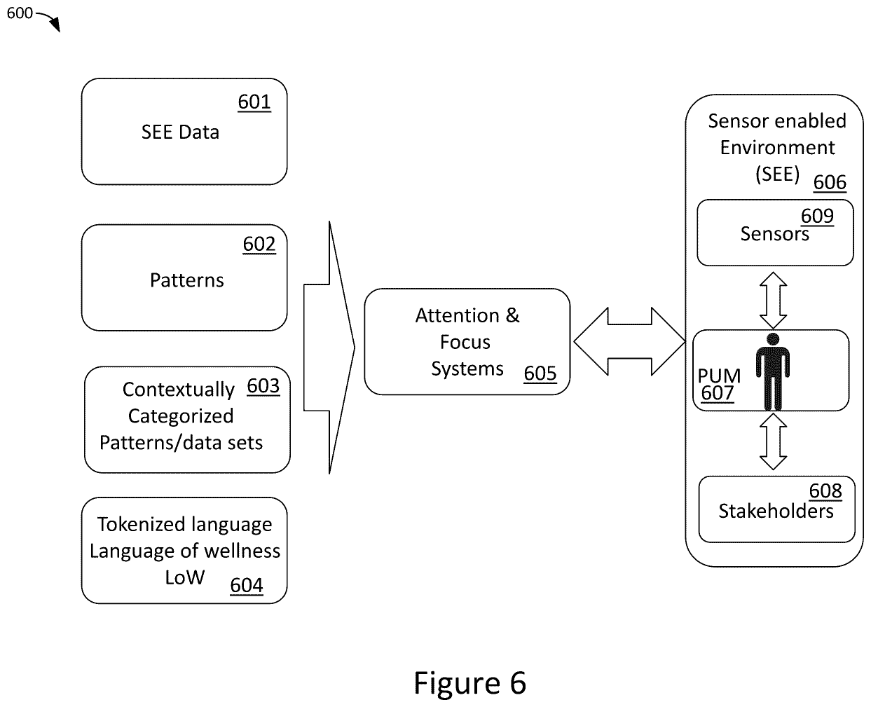

FIG. 6 illustrates a block diagram of an attention and focus dataflow 600, according to example embodiments of the present disclosure. For example, SEE data 601, patterns 602, contextually categorized/classified patterns/data sets 603, or tokenized one or more care languages, for example language of wellness (LoW) 604 may be communicated, in whole or in part, to one or more attention and focus systems 605. Such system may interact with a SEE 606, including with sensors, devices, or systems 609, one or more PUM 607, or one or more stakeholders 608 in any arrangement.

In some embodiments, a care hub or care processing system may include a response module which may communicate with one or more other systems elements. The response module may generate candidate responses to current or predicted states, events, or actions of a PUM in a SEE. This may include response candidates that may calibrate or configure the one or more sensors, devices, or systems that are deployed or present in a SEE. These response candidates may include data sets, pattern frameworks, patterns, state, configurations, calibrations, one or more languages, or other elements in any arrangement.

In some embodiments, this may include the use of one or more tokenized language models, for example a monitoring language such as language of wellness (LoW) which may be expressed as set of tokens. For example, a set of X tokens comprising token A/B/C may equate to a monitoring situation. Further languages may be employed, such as quality of life (QoL) or response candidate (RC), which may also be tokenized representations of actual or potential states, actions, events, or situations of one or more PUM in a SEE.

In some embodiments one or more AI/ML modules may contribute to determining one or more response candidates that include characteristics, including metrics of one or more languages representing the quality of life (QoL) of the PUM in a SEE. In this manner such modules may operate to evaluate a potential of response candidates to impact the quality of life of a PUM.

For example, AI/ML modules employing techniques such as retrieval augmented generation (RAG) may be employed to, at least in part, determine potential response candidates which may include use of digital twins or physics engines with which to assess or evaluate one or more contextual behaviors of a PUM in a SEE.

In some embodiments there may be one or more response frameworks which represent particular responses to likely, well known, or predicted events affecting a PUM. For example, there may be one or more fall response frameworks which may be instantiated depending on a severity of a fall as detected by the one or more sensors, devices, or systems of a SEE, which for example may be represented by one or more patterns or states. These response frameworks may be stored in one or more repositories and may be made available to multiple PUM domiciled in multiple SEE. For example, there may be a response candidate that is configured for immediate response, such as calling 911 or other emergency services. There also may be one or more standardized response frameworks, including standardized responses for known PUM adverse situations, such as falls.

In some embodiments, response frameworks may comprise one or more data sets generated by one or more sensors, devices, or systems deployed or present in a SEE, patterns of such data sets, state data, one or more categorized data sets, including spatial, temporal, contextual behavior, or wellness or other data, including calibration or configuration data in any arrangement.

In some embodiments, one or more pattern frameworks including, at least in part, data sets or patterns generated by the one or more sensors, devices, or systems of a SEE may be aligned with event pattern frameworks representing a potential health and wellness event that may impact a PUM. Such an alignment may be stored in one or more repository where one or more AI/ML modules may determine, at least in part and using, for example, one or more digital twins or one or more physics engines, likely correlations or alignments of the pattern frameworks and event frameworks as each of these is populated by the data sets or patterns generated by the one or more sensors, devices, or systems of the SEE. This determination may include the use of game theory where, for example, one or more strategies may be employed or identified to establish alignment, correlation, or causation. These determinations may, in some embodiments, be stored in one or more repositories.

In some embodiments, determinations may form one or more response frameworks where, upon population of the one or more pattern frameworks or event frameworks with the data sets or pattern generated by the one or more sensors, devices, or systems of a SEE, one or more thresholds, metrics, or other variables may be satisfied such that the care hubs or care processing systems may initiate one or more response candidates based at least in part on the one or more response frameworks.

In some embodiments, one or more care hubs or care processing systems may include the use of quality of life (QoL) metrics which may include impact analysis for selection or deployment of one or more response candidates.

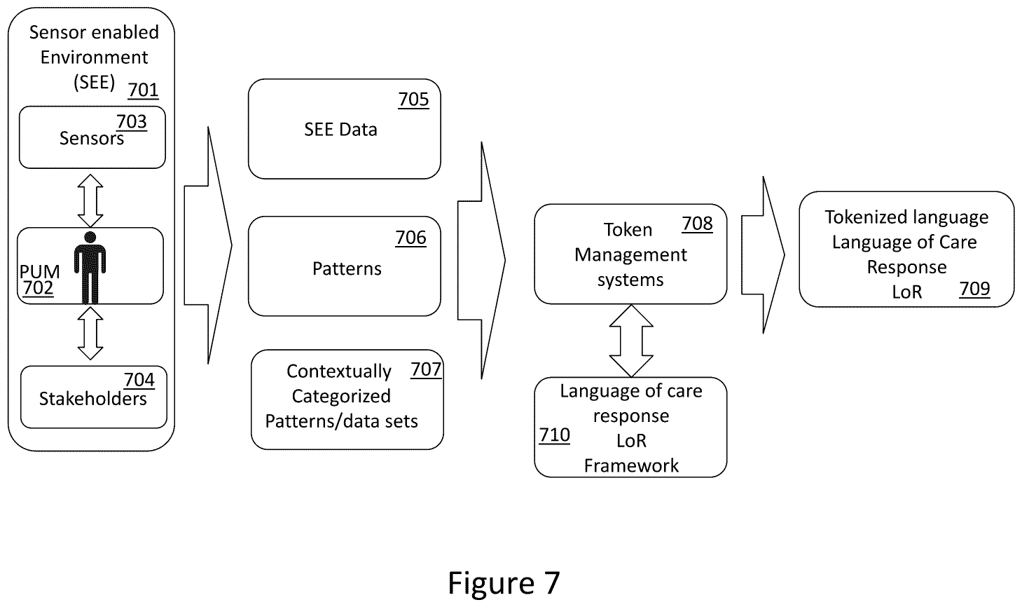

FIG. 7 illustrates a block diagram of a token management dataflow 700 for responses, according to example embodiments of the present disclosure. For example, a SEE 701 including one or more PUM 702, one or more stakeholders 704, or one or more sensors, devices, or systems 703 employed for monitoring in the SEE 701 may generate SEE data 705, patterns 706, or contextually categorized/classified patterns/data sets 707 which are communicated to one or more token management systems 708 which may employ a care language framework, for example a language of care response (LoR) framework 710 may generate a tokenized care language, for example a language of care response (LoR) 709.

Using a combination of an SEE including embedded or present sensors or devices, fixed or mobile, in combination with one or more physics engines configured to represent measurements and data sets generated by such sensors or devices, a remote monitoring capability may be instantiated using, for example, one or more virtual reality capabilities, where the SEE and the PUM therein may be visualized and represented within such a virtual environment. In some embodiments this may include one or more representations of a PUM, for example as a digital twin. The use of such digital twin of a PUM may include actual or predicted behaviors, where such PUM actual sensed behaviors may be compared, for example, with predicted behaviors based at least in part on pattern frameworks or tokenized behavior representations including one or more categorizations.

In some embodiments, such monitoring may be represented on a screen, such that a virtual PUM representation may be monitored within the SEE and the PUM's behaviors are, at least in part, evaluated by one or more AI/ML system so as to, at least in part, determine any variations that may indicate a care or wellness event.

In some embodiments, such monitoring may use, for example, cameras, microphones, haptic, or other fixed, carried, or worn sensors and devices, where the measurements and data of such sensors or devices may provide data in a format that protects privacy of the PUM, for example using tokens to represent such data which may be used to configure one or more representations of the PUM in one or more virtual environments.

In some embodiments where monitoring of the representations of the PUM deviate, vary, or in other manners indicate that a care and wellness event may be forthcoming or occurring, the monitoring may invoke raw data feeds from the SEE, for example, configuring a camera to provide live images, configuring a microphone to provide live audio, or other approaches of providing live data from the SEE. Such configuration may include alerting the PUM or other stakeholders as to the change in configuration of the sensors or devices.

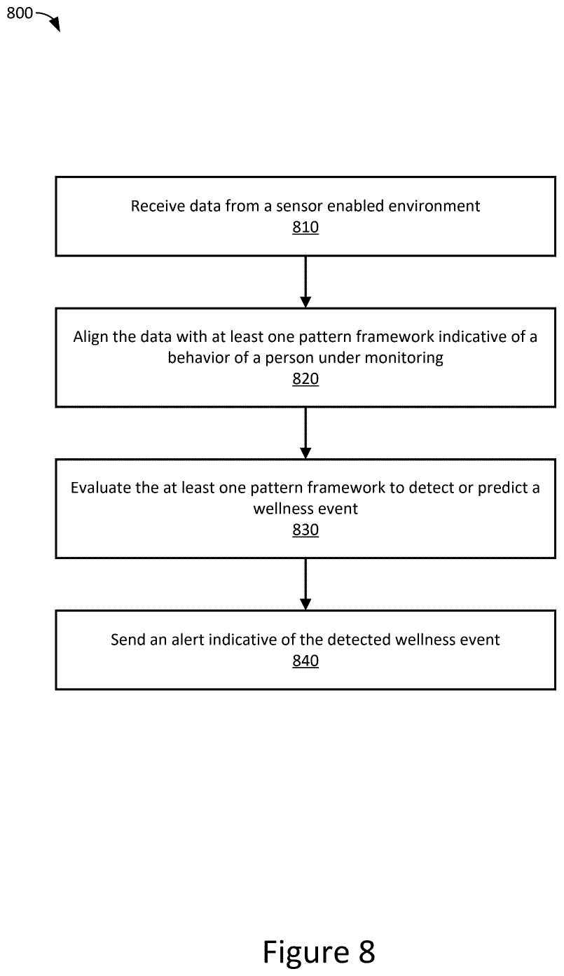

FIG. 8 illustrates a flowchart for an example method 800 for monitoring a SEE, according to example embodiments of the present disclosure. It will be appreciated that the method 800 is for illustrative purposes only, is not intended to be limiting, and is presented with a high degree of generality for ease of understanding. It will therefore also be appreciated that steps of the method 800 may themselves comprise several sub-steps, that steps of the method 800 may be excluded, and that additional steps not illustrated may be included in actual embodiments of the method 800.

At block 810, an example AI/ML model receives data from a sensor enabled environment. For example, a care hub 302 executing a convolutional neural network trained on historical monitoring data 313 from Alzheimer's patients may receive temperature, haptic, audio, and video data from an Alzheimer's patient's home.

At block 820, the example AI/ML model aligns the data with at least one pattern framework indicative of a behavior of a person under monitoring. For example, the AI/ML model may deduce from the temperature data that a stove has been turned on in the kitchen, which may align with a cooking framework. The AI/ML model may also determine from the video and haptic data that the Alzheimer's patient has entered a bed, which may align with a sleeping framework.

At block 830, the example AI/ML model evaluates the at least one pattern framework to detect or predict a wellness event. For example, upon determining that the cooking and sleeping frameworks are active simultaneously, the AI/ML model may determine that the Alzheimer's patient has left the stove on and gone to bed, thus indicating a memory lapse event that could escalate to injury if left unaddressed.

At block 840, the example AI/ML model sends an alert indicative of the detected wellness event. For example, the AI/ML may notify one or more monitoring individuals, first responders, the Alzheimer's patient themselves, family members, or any other stakeholders.

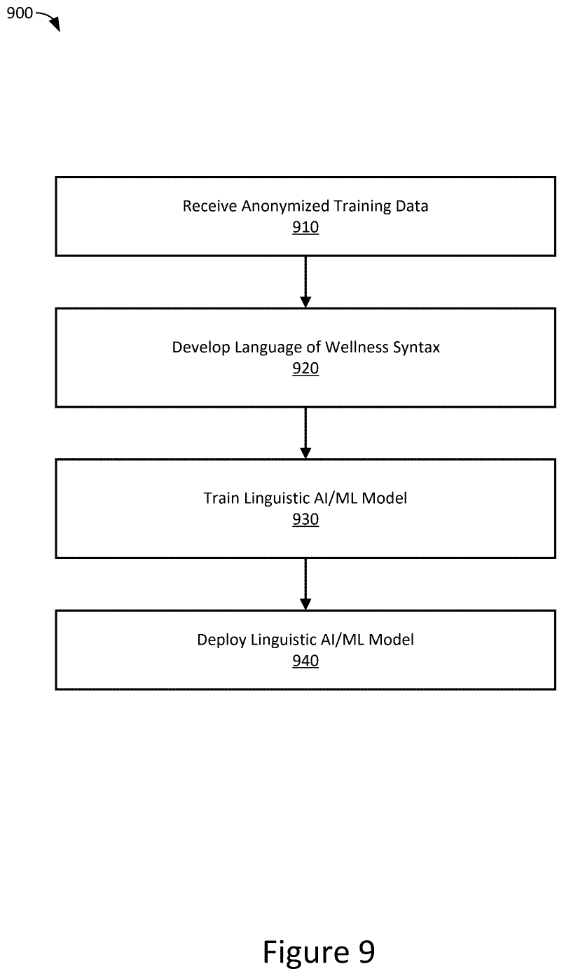

FIG. 9 is a flowchart of an example method 900 for generating and maintaining a linguistic AI/ML model for machine learning for aggregating and evaluating data from a sensor enabled environment, according to embodiments of the present disclosure.

In various embodiments, the linguistic AI/ML model generated and maintained per method 900 may be provided as a “hub” AI model for use in particular setting (e.g., a first SEE for monitoring a first PUM) after being trained initially on data not related to that particular setting. Accordingly, a linguistic AI/ML model may act as a first stage before localization or calibration tunes the model for the particular setting, or may be used in a generalized setting where the environment may be in flux, or the identify of the PUM may be in flux. For example, a linguistic AI/ML model may be used in a short-term care facility, where insufficient data may be present to tailor the model to any one PUM during an expected stay in the facility. For example, in a newly constructed or renovated SEE, insufficient data may exist regarding the layout or capabilities of the sensors, and a linguistic AI/ML model may be able to be used without need for further calibration.

Accordingly, the inventors have found several unexpected benefits of using a linguistic AI/ML model that is normally designed for handling language processing tasks, in a novel scenario for healthcare to allow for more rapid deployment, reduced training dataset size requirements, greater privacy in personal data used in the training process, compared to conventional AI/ML models used in this field. These benefits, and others that will be recognized by those skilled in the art, can be realized by treating actions, behaviors, and events identified in the SEE as semantic elements in a language of wellness (LoW), which has its own syntax and grammatical rules. When a BHWS event disobeys one or more of these rules, either predictively, or reactively, the linguistic AI/ML model may generate and transmit an alert to one or more stakeholders to mitigate or prevent harm from such actions.

For example, much like the syntax of a human language can be used to identify when a sentence is mis-constructed, the LoW syntax can identify when a data stream or a series of events from a SEE is malformed, which can be used as an alert condition in monitoring a health condition for a PUM (e.g., for treatment or prophylaxis thereof). For example, with reference to the English sentence of “The lazy brown dog is mine, but the quick fox is not”, the vocabulary used, order, declension, conjugation, and use of pronouns affect the meaning conveyed, and a native speaker will recognize that several other syntactically appropriate ways to express the same facts exist. For example, the first sentence may also be represented as “The quick fox is not mine, but the lazy brown dog is” or “The fox is not mine and the dog is, wherein the fox is quick, the dog is lazy, and the dog is brown.” However, even using the same words as the first sentence, when syntax is disobeyed, the reader will note that the sentence is malformed and something is amiss (e.g., the alphabetized “Brown but dog fox is is lazy mine, not quick the the”). Similarly, the LoW syntax provides a framework to judge when well-formed data and event determinations (e.g., valid heart rate data and valid determinations of hearth health events) combine to describe a situation in the SEE that warrants further investigation or generating an alert to a stakeholder or the PUM.

To develop the linguistic AI/ML model and the related syntax for the LoW, method 900 begins at block 910, where a central service, such as a model generation system receives anonymized training data from a plurality of SEEs related to monitoring various PUM according to various associated HCP for those PUM. In various embodiments, these data are tokenized, so that tags identifying features of the data can be read without the need to decrypt all of an associated data set (e.g., to identify data relevant to a set of criteria, such as certain health conditions, certain locations of SEEs, demographic data for a type of PUM for whom the data were gathered, etc.). In various embodiments, the data are stripped of personally identifiable information or such information remains encrypted so that when data from multiple SEEs are received, the aggregated data are anonymized and information related to a particular SEE or particular PUM cannot be determined from the aggregated data set or otherwise linked back to the particular SEE or particular PUM. In various embodiments, the tokenized data identify whether a behavioral, health, wellness or safety event occurred, whether an alert was generated for a such event, whether the alert was a false or true positive or a false or true negative, and combinations thereof.

In some embodiments, a central service periodically or in response to a behavioral, health, wellness, or safety (BHWS) event occurring (e.g., a deployed linguistic AI/ML model detecting alert conditions) receives updated information to continue improving the models with. For example, a central service can receive from the computing device, tokenized alerts of BHWS events affecting the particular PUM identified via a deployed instance of the AI/ML model and update one or more data sets with the alert and data carried therein. Which data sets are updated may be based on one or more categories of the BHWS event or classifications of the PUM that are readable in the tokenized alert matching or corresponding to one or more categories for training/retraining generalized AI/ML models for use PUMs having similar categories of health conditions monitored for or belonging to a similar category of PUM. For example, a tokenized alert can indicate that the token relates to a health alert of a fall affecting a person identified as between 60-80 years old, and is added to two data sets-one for persons who have fallen, and one for persons between 60-80 years old. The encrypted data in the tokenized alert can then be anonymously aggregated with the other data in the data set (e.g., reported data from sensors in the SEE, behaviors or activities identified as occurring prior to the fall, whether the fall was a false positive or true positive, whether the sensors and AI/ML model missed identifying the fall (e.g., a false negative), whether the AI/ML model correctly predicted a fall occurring and helped mitigate or preemptively alert for a potential fall, etc.).

Accordingly, the set of training data may be received from at least one SEE, and is related to BHWS events affecting at least one PUM. These PUM can be associated, for example bound through tokenized, cryptographic, reference or embedding techniques, with at least one corresponding SEE, and each SEE corresponds to at least one PUM.

In some embodiments, the central service, for example, via one or more machine learning models, can align the training data with at least one pattern framework indicative of a behavior of a person under monitoring to develop the tokens of the BHWS events. For example, the AI/ML model may deduce from the temperature data that a stove has been turned on in the kitchen, which may align, in whole or in part, with a cooking framework. The AI/ML model may also determine from the video and haptic data that the Alzheimer's patient has entered a bed, which may align with, for example, a sleeping framework. In some embodiments, the training data are received pre-aligned and are provided as tokens of the various BHWS events.

At block 920, the central service develops the LoW syntax for the linguistic AI/ML model. In various embodiments, the linguistic AI/ML model may include one or more large language model (LLM), retrieval augmented generation (RAG) model, a private large language model (PLLM), or a specialized language model in any arrangement.

In some embodiments, an AI/ML module may develop a grammar or syntax which can be machine or human readable that represents, in part or in whole the relationships between the “words”, for example represented by one or more tokens. In some embodiments these tokens can represent the data sets generated by the one or more sensors, devices and/or systems of the SEE.

In some embodiments the development of such grammar and syntax by the AI/ML module can be dynamic, for example in response to the state of a PUM in a SEE. In this manner the AI/ML module, which can form part of a central or distributed service, may operate with on a non-human interpretable grammar and syntax that represents the measured and observed state of the PUM to identify, validate or determine a behavioral, health wellness, or safety condition, including quiescent state, of the PUM. In some embodiments, this non-human interpretable grammar and syntax may be converted by, for example a further service, including the use of one or more LLM, RAG or other AI/ML systems into a human readable grammar and syntax that enables a human stakeholder, for example a carer, to interpret the operations of the AI/ML module and the output thereof, for example an alert, event or other communication.

In many circumstances the use of an AI/ML system to generate a grammar and syntax representing the relationships, including connections, between identified and potentially classified and/or categorized data sets, for example those represented by patterns, behaviors, events or other representations of the state of the PUM in a SEE can enable a more timely, efficient, including computationally, accurate and responsive monitoring of a PUM in a SEE.

In some embodiments such grammar and syntax can form, in whole or in part a Language of Wellness (LoW), where for example an AI/ML system may generate a grammar and syntax that is specific to an individual PUM, a group thereof having one or more common attribute and/or a broad diaspora or PUM in any arrangement.

As will be appreciated, linguistic models are typically designed to process natural human language, and are trained on text or other human generated input in a particular language. For example, a first model may be trained on English, a second model on Japanese, and an nth model on an nth language. The present disclosure proposes that the benefits of linguistic models be applied in a new fields and in unexpected manners by rendering actions and events rendered into a language of wellness (LoW). Accordingly, the central service develops the LOW and the associated syntax by which to evaluate the BHWS events (e.g., tokenized collections of sensor data and associated analyses) occurring the in the SEE, and thereby permits the use of linguistic models in an unexpected manner, which improves and expands the underlying capabilities of the computing devices on which the linguistic AI/ML model is eventually deployed.

In some embodiments, a central service can analyze one or more sequences of events to develop the language of wellness (LoW). This language, in common with other languages, may have a set of expressions, a syntax, a grammar, and a set of semantics. Such syntax may be influenced by various spatial, temporal, and pattern-like behaviors, which form the semantics of the language. For example, an event of brushing teeth may be frequently seen in proximity to a sleep event (e.g., on waking or prior to going to bed), but only after a meal event (e.g., not within X minutes before a meal). These sequences or arrangements may be set manually, or identified organically by the central service based on the how the “words” or “phrases” of the various events are put together. In some embodiments, one or more sensors, devices or systems may contribute to such sequences and/or arrangements, where for example a sensor or device provides a “word”, for example represented as a token, that represents an event, for example using a toilet, brushing the teeth and the like. In this example, one sensor, for example, an audio microphone may recognize the sounds of such activity as matching a classified or categorized activity and generate a “word” forming part of the LOW, which can be passed to one or more service. In this example, such sensor may also communicate with other sensors to validate or increase the certainty of the recognition, for example through monitoring water flow, mechanical impact or other observable indicia.

In various embodiments, the syntax may be temporal, so that a group of events is examined in time or in a time window. For example, an event of waking that occurs at time t1, an event of eating (or skipping) breakfast at time t2, and an event of a low/high blood sugar event at time t3 can be understood as a temporally ordered sequence with a syntax from t1-t3. As will be appreciated, temporal syntax may include events occurring simultaneously or with overlapping time ranges. For example, an event of the stove being on from time t1-t4 may be understood with reference to a PUM eating breakfast from time t3-t8.

In various embodiments, the syntax may be spatial, so that a group of events is examined in space or in a spatial region. For example, an event occurring in a bedroom can be understood with respect to an event occurring in a kitchen (e.g., a first PUM waking while a second PUM cooks breakfast). For example, an event of a fall occurring may be understood with a spatial reference to a bathroom (e.g., slipping on a wet floor) differently than a fall occurring in a living area (e.g., tripping on a loose carpet) and differently than a fall occurring in a sleeping area (e.g., falling out of bed).

In various embodiments, the syntax may be relational, so that a group of events is examined with respect to the identity or types of events in that group. For example, events of teeth brushing and showering may be identified as being syntactically related, as are events of showering and falling, whereas events of teeth brushing and a falling are not. In some environments such relationships may be represented in one or more graph databases or topological models.

As will be appreciated, the syntax may include various combinations of different temporal, spatial, and relational constructions and interrelationships. These relationships may be used predictively by the end user, to identify when a behavioral, health, wellness, or safety incident of a dangerous, medically significant, or undesirable event is expected to occur and potentially ameliorate or prophylactically avoid the event occurring. For example, if the syntax predicts that a PUM having skipped breakfast is at risk for a low blood sugar event, the linguistic AI/ML model may produce a predictive alert for the PUM to eat something sugary to avoid the low blood sugar event.

These relationships may also be used reactively by the end user, to identify when an event has occurred that deviates from an expected syntax, but may constitute a behavioral, health, wellness, or safety incident of a dangerous, medically significant, or undesirable event. For example, when the syntax indicates that the PUM should perform action 1, action 2, or action 3 after a particular series of BHWS events is observed, but a third action occurs, the linguistic AI/ML model may produce a reactive alert for the PUM. For example, a PUM who leaves a bedroom in the morning and enters a kitchen, may be expected to remain in the kitchen (e.g., to cook and eat), return to the bedroom (e.g., to retrieve a forgotten item), or head to a bathroom (e.g., for morning toilet). However, if the PUM repeatedly leaves and returns to the bedroom, such as during a Altheimer's event, the AI/ML model may identify that the repetitive behavior is unusual and disobeys a syntactical rule developed in the syntax used by the AI/ML model, and therefore a reactive alert is generated.

At block 930, once the LOW and the syntax is developed, the central service trains the linguistic AI/ML model using the syntax of this language, as one of skill in the art would understand to train a typical linguistic AI/ML model. As will be appreciated, linguistic AI/ML models operate on prompts and produce outputs according to the corpus on which they were trained and the prompt. For example, by developing the LoW, the central service can provide a model that accepts as part of the prompt one or more of: the tokens of events occurring in the SEE, raw (encrypted or unencrypted) sensor data from the SEE, details of the PUM or SEE, lists of events occurring in the SEE or affecting the PUM, and the like. In some embodiments, the prompt may include provision of an ongoing data stream of raw sensor data from one or more sensors in the SEE to a hub-based linguistic AI/ML model. In some embodiments, the prompt may include provision of an ongoing token stream of processed sensor data as tokenized events from one or more tokenization services or models that process the sensor data from sensors in the SEE, which therefore provides tokens to a hub-based linguistic AI/ML model to monitor ongoing events in the SEE. Accordingly, the prompt may provide the decryption keys and ongoing data or analysis from sensors disposed in the SEE for continued analysis by the linguistic AI/ML model, or may provide a one-time set of sensor data and analyses for a one-time analysis by the linguistic AI/ML model.

At block 940, the central service deploys the AI/ML model. In various embodiments, the central service hosts the AI/ML model as a “hub” AI that the computing devices located (remotely from the central service) transmit prompts to, so that predicted or analyzed results are returned to the requesting computing device without having to host an instance of the linguistic AI/ML model itself or transmit large amounts of sensitive data for detailed analysis on a remote system. Accordingly, the linguist AI/ML model acting as a hub can improve data security and reduce network traffic, among other benefits, while offering high-quality analysis of the PUM in various locations. In some embodiments, this can include the use of tokens as, in part or in whole, the communications amongst and between the AI/ML model, one or more SEE or one or more PUM or other stakeholders and the devices thereof.

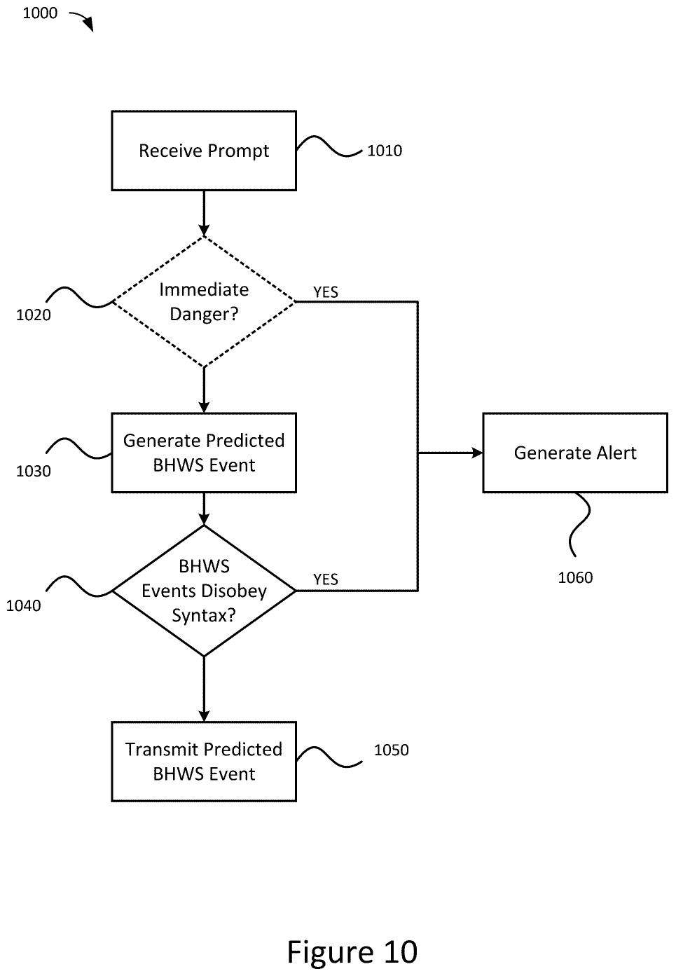

FIG. 10 is a flowchart of an example method 1000 for deploying and using a linguistic AI/ML model for machine learning for aggregating and evaluating data from a sensor enabled environment. Although generally discussed in the context of deploying the linguistic AI/ML model in a hub AI configuration, the present disclosure contemplates that instances of the “hub” linguistic AI/ML model may be deployed to various computing devices for localized use as “edge” AI models or across multiple platforms as “mirrors” of the hub linguistic AI/ML model. In various embodiments, the deployed linguistic AI/ML model may be the model generated according to method 900, which is configured to process prompts according to a language of wellness (LoW) syntax using one of various formats of language-based AI/ML models (e.g., RAG, LLM, PLLM).

At block 1010, the linguistic AI/ML model receives a prompt. In various embodiments, the prompt is received from a computing device associated with a particular SEE (and one or more particular PUM or other stakeholder), and which may be located remotely from the server or computing device on which the AI/ML model is deployed, and may include one or more BHWS events.

In some embodiments, the prompt may include provision of an ongoing data stream of raw sensor data from one or more sensors in the SEE to a hub-based linguistic AI/ML model. In some embodiments, the prompt may include provision of an ongoing token stream of processed sensor data as tokenized events from one or more tokenization services or models that process the sensor data from sensors in the SEE, which therefore provides tokens to a hub-based linguistic AI/ML model to monitor ongoing events in the SEE. Accordingly, the prompt may provide the decryption keys and ongoing data or analysis from sensors disposed in the SEE for continued analysis by the linguistic AI/ML model, or may provide a one-time set of sensor data and analyses for a one-time analysis by the linguistic AI/ML model.

In some embodiments, the prompt may include or reference (e.g., via a hyperlink) a health care plan (HCP) so that the linguistic AI/ML model is provided for generating alerts, predictions, and recommendations for the treatment or prophylaxis of a health condition indicated for the particular PUM in the HCP. Such a HCP may be encrypted and decrypted using a different key than other elements of the prompt, or may be included in a generalized sense in an unencrypted state. For example, a prompt may request the AI/ML model to generate one or more predicted BHWS events for a patient of a specified age range and health condition given one or more tokens of events monitored in the SEE or data streams from the SEE.

In various embodiments, the presently described linguistic AI/ML model allows for greater data security, as tokenized behaviors included in the prompts can remain encrypted and anonymized, and constitute a smaller amount of data needed to be transmitted for analysis than in conventional systems.

The present disclosure contemplates that a BHWS can describe various incidents that may be classified as one or more than one of a behavioral incident, a health incident, a wellness incident, or a safety incident for various PUM, and may be determined by interactions among several such incidents or events to describe an emergent event. Additionally, BHWS incidents may include both “positive” and “negative” incidents, including for example those with one or more metrics characterizing such incidents, such as risk metrics and the like, or incidents that can be characterized in multiple manners or as or as multiple ones of behavioral incidents, health incidents, wellness incidents, or safety incidents. For example, a dementia flare-up may be classified as both a health event and a behavioral event, and may include several other BHWS events that, when combined, describe the BHWS event as a dementia flare-up event in addition to or instead of as the individual events thereof. Additionally or alternatively, as an inverse to a dementia flare-up event, a lucidity-break (e.g., a positive vs. negative event) may be monitored and alerted for using the same or different sensors and the same or different alerting conditions.

For example, a first PUM may be monitored for the presence of the behavior of “eating breakfast”, while a second PUM may be monitored for the absence of the behavior (e.g., “skipping breakfast”), which may be classified as a behavior incident as well as a health incident, wellness incident, or a safety incident depending on the HCP for the PUM, and the occurrence or timing of the BWHS incident may be treated positively or negatively according to the HCP. For example, both the first and second PUM may need to avoid eating breakfast due to medications needing to be taken on an empty stomach, so “eating breakfast” may be handled as a negative incident (e.g., resulting in an alert), whereas “skipping breakfast” may be handled as a positive incident (e.g., not resulting in an alert). In a contrasting example, both the first and second PUM may need eat breakfast due to blood sugar requirements, so “skipping breakfast” may be handled as a negative incident (e.g., resulting in an alert), whereas “eating breakfast” may be handled as a positive incident (e.g., not resulting in an alert).

In various embodiments, BWHS incidents (or events) may be triggered via detection of a one-time or an ongoing condition in the SEE or affecting the PUM. For example, a BWHS incident may monitor whether a PUM has fallen, and is indicated in response to a sound, impact, positional sensor, or combination thereof indicating that the PUM has fallen. In another example, a BWHS incident may monitor whether the PUM is affected by a tachycardia condition, and is indicated in response to a heart rate monitor indicating a heart rate above a threshold rate for at least a threshold time (e.g., to avoid false positives from day-to-day excitements).

In various embodiments, the prompt may include a class or other details of the PUM or a healthcare plan (HCP) for the PUM, which may include various non-personally identifiable information relevant to the health of the PUM. For example, the prompt may identify an age, gender, reason for monitoring, available sensors associated with the PUM, available sensors available in the SEE, etc. In various embodiments, the linguistic AI/ML model and the requestor may agree on using a unique identifier so that multiple prompts can be linked over a period of time for longitudinal analysis of the PUM or SEE, without linking the results in an identifiable manner to third parties with the PUM or the SEE.

In various embodiments, the prompt may include one or more tokenized observations of the SEE or PUM. In various embodiments, the tokenized observations may omit some or all of the underlying sensor data used to reach the identification of a particular event included in the prompt. For example, rather than sending the raw (encrypted) data that lead to a determination that a PUM suffered a fall event at time T at location L, the tokenized observation may indicate that a fall occurred at time T at location L with no further data attached. In an additional example, the tokenized observation may indicate that a PUM suffered a fall event at time T at location L, and include some set of the sensor data to provide additionally context for syntactical analysis of the event (or surrounding events). In various embodiments, the linguistic AI/ML model may request that a prompt include or exclude certain data and query the requestor for such data if the data are desired.

At block 1020, the linguistic AI/ML model optionally determines whether an immediate danger state is identified in the BHWS included in the prompt. In various embodiments, an immediate danger state is identified based on a current state of the PUM or SEE being associated with a currently detected condition identified with an alert condition. For example, temperature of over X degrees Fahrenheit in the SEE may be generally present to indicate a fire in the SEE, which, when detected, is classified as an immediate danger state that causes an alert to be generated. In an example, detection of an open window may be classified as an immediate danger state for a PUM with dementia (e.g., for an increased risk of exit from the SEE) if the prompt indicates that the PUM is a dementia patient, but not if the prompt indicates the PUM is otherwise healthy. In an example, detection of a PUM on the floor of the SEE for at least X minutes be classified as an immediate danger state (e.g., indicative of a fall or other BHWS event) unless the prompt indicates that the PUM frequently engages in activities that occur while lying on the floor (e.g., yoga, stretching, prescribed/preferred time lying on a hard surface, etc.), and the linguistic AI/ML model may ignore such data as not indicative of an immediate danger state in the particular case.

When the computing device that transmitted the prompt indicates that such immediate danger states are handled locally (e.g., via a watchdog application), the linguistic AI/ML model may omit performing block 1020, or may include any alert generated per block 1060 in a transmission to the prompting computing device to handle locally. When an immediate danger condition is detected, method 1000 proceeds to block 1060 in addition or alternatively to proceeding to block 1030.

At block 1030, the AI/ML model generates one or more predicted BHWS events based on the prompt. In various embodiments, the provision of current and historical BHWS events allows the AI/ML model to predict potential future BHWS events using the syntax of the LoW. In various embodiments, the linguistic AI/ML model identifies the current state of the particular PUM and the particular SEE based on the prompt, and identifies one or more digital twins of the PUM, the SEE, or other entities in the SEE to simulate what the twinned entity will do next to generate the predicted BHWS event.

In various embodiments, each digital twin incorporates specifications of the capabilities of the entity that the digital twin represents. Each digital twin incorporates the physical characteristics of the entity and represents the state of the entity within a simulation of the environment. The interactions between digital twins provide an accurate and timely predictive representation of the interactions between the entities, which can be used to generate candidate next states over a plurality of iterations, where more-likely candidate next states are simulated more often than less-likely candidate next states.

In various embodiments, digital twins can represent the care and wellness state of a PUM and the environment with sufficient fidelity so as to be used in predictive analytics, including the use of AI or machine learning, for the care and wellness benefit of the PUM. In many circumstances the digital twin can be one of a set of digital twins representing a set of PUM that have a common set of care and wellness characteristics, such as the same or similar HCP and the operating patterns and pattern elements thereof. Accordingly, the digital twin can comprise a dynamic tokenized representation of quiescent or active behaviors of a specific PUM in a specific environment or represent a generalized model of a PUM in a generalized environment, which may be localized or used as-is. The tokenized behaviors can identify or name the observed behavior, thereby labeling the token (of Bevoken) as corresponding to a specifically identified behavior, and keeping the data used to reach that identification encrypted.

The degree of disclosure of the data sets pertaining to a PUM to a digital twin may be sufficient for the digital twin and any associated analytic or predictive processing to be able to undertake effective predictive, trend, underlying care framework identification or other care and wellness benefit processing. This can be achieved in a number of different manners, employing differing embodiments, for example the tokens may include a set of specifications that can be interpreted by a suitably authenticated and authorized digital twin that can access, potentially on a time or purpose limited basis the data that are deemed private by a PUM for a specified purpose. This type of disclosure may be agreed by a PUM or their authorized proxy in advance. The data received by the digital twin may be expunged after the appropriate analytics or processing has been undertaken. In some embodiments, the digital twin may operate as a proxy for a PUM, such that all data are available to a digital twin, and the digital twin acts as the privacy guardian of the PUM, enacting and enforcing the privacy choices of the PUM. In a further embodiment, there may be tokens that are digital twin specific and include further specifications determining the use, propagation, or configuration of the tokens and the digital twin operating upon them. Accordingly, the digital twin, through one or more configurations, retains a trust relationship with the PUM, such that the data a PUM deems private remain private, and the digital twin can operate to support the care, wellness or safety monitoring of the PUM to the benefit of the PUM.

In various embodiments, a plurality of digital twins can be used to simulate a single PUM with different configurations or monitored behavioral, health, wellness or safety (BHWS) conditions so that each digital twin instance can produce different predictive results. For example, a first digital twin may be configured to monitor for the PUM falling, and is configured to represent the PUM in a distracted state (e.g., based on movement patterns trained on sleepy, feverish, or inattentive historical users), a second digital twin may be configured to monitor for the PUM falling, and is configured to represent the PUM in an alert state (e.g., based on movement patterns trained on rested, healthy, or assisted historical users), and a third digital twin may be configured to monitor the PUM for heart attacks (e.g., based on historical conditions of users when struck with a cardiac event).

In various embodiments, the digital twins may use, or be used in conjunction with, one or more game theory models to, at least in part, identify potential behaviors of the PUM expressed as data sets representing patterns or to rank those behavior sets into one or more ordered arrangements. In various embodiments, wherein the plurality of candidate next states can be analyzed as a Markov chain from the current state as contextual behaviors depending from the current state with the digital twins affecting the weightings of the next states in the chain from a current state.

At block 1040, the AI/ML model determines whether the BHWS events included in the prompt, received historically that can be linked to the prompt, or generated as predicted BHWS events disobey the syntax of the LoW.

In various embodiments, disobeying the LoW syntax includes disobeying an established spatial zone in the particular SEE (e.g., as a spatial rule violation), wherein the predicted BHWS is predicted to occur outside of the established spatial zone. For example, a PUM moving repeatedly between locations, or performing a tasks in a wrong location (e.g., washing dishes in a bathroom sink vs. a kitchen sink) may be identified a spatial syntax violations that may be indicative of a dementia event.

In various embodiments, disobeying the LoW syntax includes disobeying a time window (e.g., as a temporal rule violation), wherein the predicted BHWS is predicted to occur outside of the time window, wherein the time window is one of an absolute time window during a day or a relative time window from performance of a previous behavior by the PUM. For example, a PUM dressing to leave the SEE at 3 in the morning may be identified as a temporal violation of the syntax indicative of a potential escape or unauthorized activity event.

In various embodiments, disobeying the LoW syntax includes disobeying an established order for performing a first behavior relative to a second behavior (e.g., as a pattern rule violation). For example, a PUM dressing (e.g., a dressing event) before taking a shower (e.g., a bathing event) may be identified as a pattern syntax violation indicative of a potential dementia event.

In various embodiments, disobeying the one or more aspects of the LoW syntax includes disobeying a combination of spatial, temporal, or pattern violations of the LoW syntax. For example, a PUM taking a shower and not getting dressed at least X minutes after the bathing event concludes may be identified as one or both of a syntax violation according to temporal syntax and a pattern syntax.

In various embodiments, disobeying the LoW syntax includes identifying event structures that are indicative of a discrepancy in the SEE deserving of an alert, which may be independent of a BHWS event for the PUM, or related to events that would not (ordinarily) result in a PUM-based BHWS alert. For example, a PUM being identified as taking a shower in the bathroom and cooking breakfast in the kitchen simultaneously may be identified as events deserving of an alert with respect to the PUM (e.g., a unauthorized visitor in the SEE) or deserving of an alert with respect to the SEE (e.g., a sensor, clock, or network fault). In another example, when the AI/ML model identifies that the PUM has left the SEE, AI/ML model may identify a syntax violation if the PUM does not return by a certain time, an appliance activates (or remains activated) while the PUM is outside of the SEE, the sensors report another person in the SEE (e.g., as a sensor error, a model error in recognizing the PUM, an unauthorized entry), or the like.

The present disclosure is initially presented using the vocabulary, grammar, and syntax of written English; a human-comprehensible language. Accordingly, the examples given herein are provided in a human-comprehensible format, which is expected to change, but remain human-comprehensible, when the disclosure is translated into a second human language according to the vocabulary, grammar, and syntax of that second human language. The present disclosure, however, contemplates that intricacies of the LoW syntax may be at least partially incomprehensible to a human, and may represent a machine-comprehensible format for describing the structure of measured events occurring the SEE. Therefore, the AI/ML model is free to develop the LoW and LoW syntax independently of any human languages, so that adherence to or deviation from the LOW syntax enables the AI/ML model to identify relationships that cannot be clearly conveyed in a human-comprehensible language or to incorporate grammatical features absent from human language.

Because the SEE attempts to describe the events occurring the in environment within the bounds of the sensors and models deployed thereto, the AI/ML model may have data beyond what human senses can collect (and therefor beyond what human language can convey) that are expressed in the LoW. Additionally, because the sensors and AI/ML models may make incorrect assessments or otherwise report events occurring differently that what actually occurred, the LoW may include syntax and grammatical features to account for the potential differences in such measurement and observation than what actually occurred. These non-human grammatical features may include indications for overlapping or stacking confidence levels, sensors tolerances, sensor refresh rates, distances between sensors used to generate a determination, encryption status of underlying data, data/decision pathways from sensors to AI/ML models, threshold strengths for generating various alerts, as well as other features that cannot be (or would not be) conveyed using human language. Accordingly, the LoW syntax provides a machine comprehensible format by which to judge the machine-collected and machine-analyzed data (and resultant determinations) in a linguistic framework to identify adherence thereto or deviations therefrom.

In various embodiments, the determination that a syntax violation has occurred may be classified as predictive (e.g., based on the predicted BHWS event causing the violation), reactive (e.g., based on the BHWS events in the prompt causing the violation), or both.

At block 1050, the AI/ML model transmits the predicted BHWS event to the computing device form which the prompt was received, which may locally determine whether the PUM complies with or deviates from the predicted BHWS and generate various alerts, wellbeing checks, or other actions accordingly. In various embodiments, the local determinations are made based on data deemed too sensitive to transmit or store outside of a network environment for the SEE (e.g., a healthcare plan), which allows for the robust provision of AI tools via a centralized hub, while still preserving privacy of the end users.

At block 1060, the AI/ML model generates an alert. In various embodiments, the alert may be transmitted from the central service to the SEE to address an ongoing or predicted BHWS event, with various amounts of encryption or tokenization applied thereto to maintain data privacy and reduce network load, while still addressing the underlying health concerns. Additionally, one or more alerts may be generated simultaneously or in sequence to one another when block 1060 is performed. The alerts may include immediate danger alerts or syntax deviation alerts (predictive or reactive) depending on the circumstances in which the alert is generated (e.g., according to determinations made according to block 1020 and block 1040, respectively), and combinations thereof.

For example, when messaging to the SEE, the alert may be directed to the PUM or a stakeholder to determine whether an identified BHWS event actually occurred, and generate a response, which can include requesting permission for various follow up actions. For example, when monitoring a PUM for fall risk, a microphone detecting a loud sound and for example using a specialist LLM or AI/ML system configured to recognize such a sound event as a fall, and a positional sensor identifying that the PUM is in a prone position may result in a detection that the PUM has fallen. The AI/ML model may generate an alert for transmission to the SEE for a stakeholder or other caretaker present in the SEE to check on the PUM, for the PUM to self-report a status, etc. A responder to the alert in the SEE may indicate that a fall actually occurred and may positively authorize the AI/ML model in a reply to the alert to place an alert with an outside party (e.g., an ambulance service, emergency medical service provider, or non-emergency healthcare provider), indicate that a fall actually occurred and deny authorization for the AI/ML model to place an alert with an outside party, or indicate that no fall occurred (e.g., a false positive for a fall was detected).

For example, when messaging externally to the SEE, the alert may be directed to a stakeholder who has been preapproved or in indicated in the prompt for receiving certain classes of messages in certain situations. For example, a stakeholder of a relative may be contacted with an alert under condition set one, while emergency medical services may be contacted with an alert under condition set two.

In various embodiments, the external alerts are generated as tokens, which include various data sets that are encrypted, but are useable by recipients in an encrypted or partially decrypted form, and one token may include data encrypted for the exclusive use by some recipients but not others of a particular alert. For example, if an alert is generated in response to detecting that the PUM has fallen for transmission to a sets of three stakeholders of a primary care physician for the PUM, to a stakeholder of an ambulance service, and a stakeholder of a family member, the token may indicate to all three (in an unencrypted or partially decrypted state) that the PUM has suffered a fall. The alert may include data related to the lead-up to the fall and the behaviors and biometric information useful to the primary care physician, which may be of limited interest to the ambulance service or the family member (and of interest to the PUM to keep private). Similarly, the data unencryptable by the ambulance service may include address information and keycodes necessary to access the SEE (e.g., gate codes, security alarm codes, etc.) that are of limited interest to the primary care physician or the family member (and of interest to the PUM to keep private). Each stakeholder may receive the token and use, in a decrypted state, the portions that are relevant to their interested in monitoring and treating the PUM without accessing data not necessary or deemed private by the PUM for the alert-worthy situation.

Various data sets or functional models may be provided between different parties as tokens, which act to encrypt various portions of the data. For example, a token can comprise a detected data set representing behaviors of a PUM in an environment, wherein the token is encrypted using an encryption key. The tokens may contain the sensor data or may reference the data stored at the sensor. Other devices in the system or the server may make decisions on event response or escalation, without the need to access the information stored or referenced by the tokens-without decrypting the data, the token is deemed sufficient evidence of a determination or detection based on the data. Other devices within the system may obtain the data associated to the token and use those data to, for example, enhance the event detection accuracy or to confirm the event. For example, a device may interpret a combination of acceleration and change in altitude from sensors as a “fall” event for a PUM, and issue a token associated with the sensors' data and send that event token to the server and to a nearby edge device. While the server may trigger a notification to a call center or to smart speaker app to initiate a conversation with the PUM, the nearby edge device may use the token (and not the full set of data that resulted in the data), combined with its identification or other authorization key, to request the event data from the device and use it to confirm or add accuracy to the fall event, by combining the sensor data with data from its own sensors, for example audio signals from a microphone or microphone array, or output signals from one or more Frequency-Modulated Continuous-Wave (FMCW) radar sensors.

In some embodiments, the encryption key is selected based in part on the detected data set, the at least one stakeholder, on the person under care, a type of event detected by the environmental sensor, or is unique to a session of the person under care.

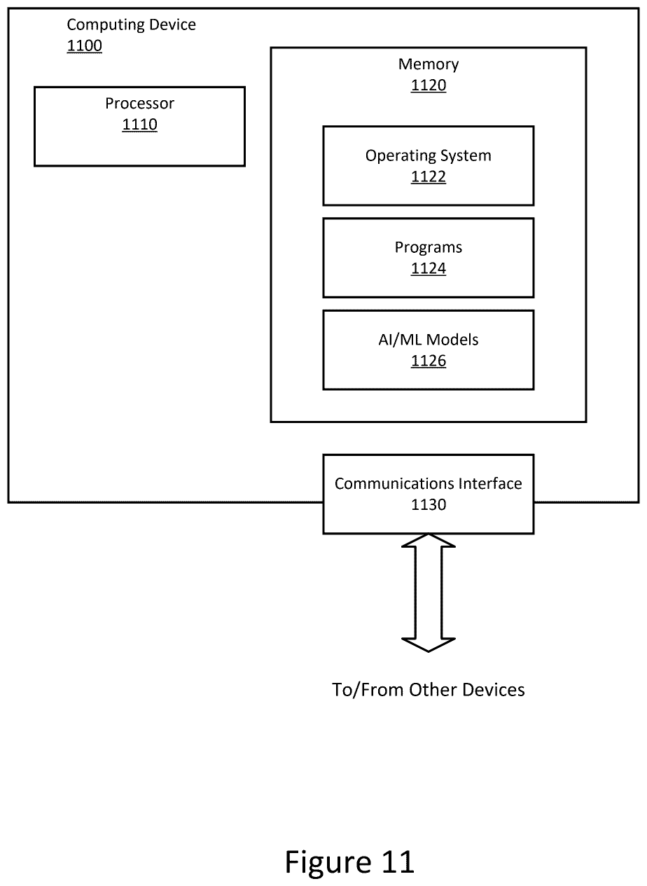

FIG. 11 illustrates an example computing device 1100, as may be used as a controller in a SEE to monitor a PUM, as part of a sensor monitoring a PUM, as part of a central or distributed service providing calibration systems for generating and curating AI/ML models for distribution to the SEEs, and the like, according to embodiments of the present disclosure. For example, the computing device 1100 may perform the operations set out in one or more of methods 800, 900, or 1000. The computing device 1100 may include at least one processor 1110, a memory 1120, and a communication interface 1130.

The processor 1110 may be any processing unit capable of performing the operations and procedures described in the present disclosure (e.g., methods 600, 700, 800). In various embodiments, the processor 1110 can represent a single processor, multiple processors, a processor with multiple cores, and combinations thereof.

The memory 1120 is an apparatus that may be either volatile or non-volatile memory and may include RAM, flash, cache, disk drives, and other computer readable memory storage devices. Although shown as a single entity, the memory 1120 may be divided into different memory storage elements such as RAM and one or more hard disk drives. As used herein, the memory 1120 is an example of a device that includes computer-readable storage media, and is not to be interpreted as transmission media or signals per se.

As shown, the memory 1120 includes various instructions that are executable by the processor 1110 to provide an operating system 1122 to manage various features of the computing device 1100 and one or more programs 1124 to provide various features to users of the computing device 1100, which include one or more of the features described in the present disclosure (e.g., method 800, 900, or 1000). One of ordinary skill in the relevant art will recognize that different approaches can be taken in selecting or designing a program 1124 to perform the operations described herein, including choice of programming language, the operating system 1122 used by the computing device 1100, and the architecture of the processor 1110 and memory 1120. Accordingly, the person of ordinary skill in the relevant art will be able to select or design an appropriate program 1124 based on the details provided in the present disclosure.

Additionally, the memory 1120 may include one or more AI/ML models 1126 that interact with, are trained by, or are curated by the programs 1124. The AI/ML models 1126 may include linguistic AI/ML models that are available for use to various SEEs as a hub AI from a central service as well as localized instances thereof for use as “edge” AI/ML models that are adjusted to reflect localized conditions in a particular SEE to track and monitor a PUM, as described herein.

The communication interface 1130 facilitates communications between the computing device 1100 and other devices, including sensors in a SEE, which may also be computing devices as described in relation to FIG. 11. In various embodiments, the communication interface 1130 includes antennas for wireless communications and various wired communication ports. The computing device 1100 may also include or be in communication, via the communication interface 1130, one or more input devices (e.g., a keyboard, mouse, pen, touch input device, etc.) and one or more output devices (e.g., a display, speakers, a printer, etc.).

Although not explicitly shown in FIG. 11, it should be recognized that the computing device 1100 may be connected to one or more public or private networks via appropriate network connections via the communication interface 1130. It will also be recognized that software instructions may also be loaded into a non-transitory computer readable medium, such as the memory 1120, from an appropriate storage medium or via wired or wireless means.

Systems, methods, and apparatuses of the present disclosure may be implemented on a variety of devices, such as but not limited to IPUs, DPUs, CPUs, GPUs, ASICs, FPGAs, DSPs, or any other device capable of processing data. Instructions for performing the same may be provided as hardware or firmware on any computer-readable medium including volatile and non-volatile forms of memory. Particular implementations of techniques of the present disclosure may have a variety of structures, including but not limited to a modular program architecture, a monolithic program architecture, on a single device, and distributed across more than one device or processor.

The present disclosure may also be understood with respect to the following numbered clauses:

Clause 1: A method, comprising: receiving training data from at least one sensor enabled environment (SEE) related to behavioral, health, wellness, and safety (BHWS) events affecting at least one person under monitoring (PUM), wherein each PUM of the at least one PUM is associated with a corresponding SEE of the at least one SEE; developing a language of wellness (LoW) syntax for a linguistic artificial intelligence or machine learning (AI/ML) model by aligning the BHWS events with a pattern framework indicative of behaviors of the at least one PUM in the corresponding SEE; training the linguistic AI/ML model based on occurrences of the BHWS events in the training data and the LoW syntax such that the linguistic AI/ML model is configured to: generate a predicted BHWS event based on a series of behaviors observed for a particular PUM in a particular SEE reported to the AI/ML model; and generate a predictive alert in response to identifying that the predicted BHWS event disobeys the LoW syntax.

Clause 2: The method of any of clauses 1 and 3-11, wherein the predicted BHWS event is predictively generated based on one or more digital twins associated with the particular PUM that simulate actions of the particular PUM within the particular SEE.

Clause 3: The method of any of clauses 1-2 and 4-11, wherein linguistic AI/ML model is configured to operate in conjunction with a physics engine associated with the particular PUM to identify when a current BHWS event or the predicted BHWS event is outside of a physical capability of the particular PUM to perform.

Clause 4: The method of any of clauses 1-3 and 5-11, wherein the training data include tokenized representations of various BHWS events, which include encrypted sensor data from the at least one SEE and an unencrypted data label identifying a type of BWHS event associated with the encrypted sensor data.

Clause 5: The method of any of clauses 1-4 and 6-11, wherein the linguistic AI/ML model is one of: a large language model (LLM); retrieval augmented generation (RAG) model; private large language model (PLLM); and a specialized language model.

Clause 6: The method of any of clauses 1-5 and 7-11, wherein the particular SEE is not included in the at least one SEE from which the training data are received, and the particular PUM is not included in the at least one PUM associated with the at least one SEE.

Clause 7: The method of any of clauses 1-6 and 8-11, wherein the linguistic AI/ML model is further configured to identify an immediate danger state based on a currently or previously observed BHWS event affecting the particular PUM in the particular SEE and to generate an immediate alert based on the immediate danger state.

Clause 8: The method of any of clauses 1-7 and 9-11, wherein disobeying the o the LOW syntax includes disobeying an established spatial zone in the particular SEE, wherein the predicted BHWS is predicted to occur outside of the established spatial zone.

Clause 9: The method of any of clauses 1-8 and 10-11, wherein disobeying the LoW syntax includes disobeying a time window, wherein the predicted BHWS is predicted to occur outside of the time window, wherein the time window is one of an absolute time window during a day or a relative time window from performance of a previous behavior by the PUM.

Clause 10: The method of any of clauses 1-9 and 11, wherein disobeying the LoW syntax includes disobeying an established order for performing a first behavior relative to a second behavior.

Clause 11: The method of any of clauses 1-10, wherein the linguistic AI/ML model is further configured to: generate a reactive alert in response to identifying that the PUM has performed or is currently performing a behavior that disobeys the LoW syntax.

Clause 12: A method, comprising: deploying a linguistic artificial intelligence or machine learning (AI/ML) model, the linguistic AI/ML model being configured to process prompts according to a language of wellness (LoW) syntax; receiving a prompt from a computing device located remotely from where the linguistic AI/ML model is deployed, the prompt including at least one tokenized behavioral, health, wellness, and safety (BHWS) event occurring in a particular Sensor Enabled Environment (SEE) associated with the computing device and with a particular person under monitoring (PUM); generating a predicted BHWS event based on the prompt; and transmitting the predicted BHWS event to the computing device as an output responsive to the prompt.

Clause 13: The method of any of clauses 12 and 14-20, wherein the linguistic AI/ML model is one of: a large language model (LLM); retrieval augmented generation (RAG) model; private large language model (PLLM); and a specialized language model.

Clause 14: The method of any of clauses 12-13 and 15-20, wherein the particular SEE is not included among training SEE from which training data used to train the linguistic AI/ML model are received, and the particular PUM is not included among training PUM associated with the training SEE.

Clause 15: The method of any of clauses 12-14 and 16-20, wherein the linguistic AI/ML model is further configured to identify an immediate danger state based on a currently or previously observed BHWS event affecting the particular PUM in the particular SEE and to generate an immediate alert based on the immediate danger state.

Clause 16: The method of any of clauses 12-15 and 17-20, further comprising, in response to identifying that the predicted BHWS event disobeys the LoW syntax: generating a predictive alert; and transmitting the predictive alert to a stakeholder associated with the particular SEE or the particular PUM.

Clause 17: The method of any of clauses 12-16 and 18-20, wherein disobeying the LOW syntax includes at least one of: disobeying an established spatial zone in the particular SEE, wherein the predicted BHWS is predicted to occur outside of the established spatial zone; disobeying a time window, wherein the predicted BHWS is predicted to occur outside of the time window, wherein the time window is one of an absolute time window during a day or a relative time window from performance of a previous behavior by the PUM; and disobeying an established order for performing a first behavior relative to a second behavior.

Clause 18: The method of any of clauses 12-17 and 19-20, wherein the linguistic AI/ML model is further configured to: generate a reactive alert in response to identifying that the PUM has performed or is currently performing a behavior that disobeys the one or more rules of the LoW syntax.

Clause 19: The method of any of clauses 12-18 and 20, wherein the linguistic AI/ML model is provided for treatment or prophylaxis of a health condition indicated for the particular PUM in a health care plan (HCP) included or referenced in the prompt.

Clause 20: The method of any of clauses 12-19, wherein the prompt includes a senor data stream from at least one sensor disposed in the SEE or a token stream from at least one tokenization service or model that processes sensor data from sensors in the SEE.

Clause 21: A system, comprising: a processor; and a memory, including instructions that, when executed by the processor, perform operations as described in any of clauses 1-20.

Clause 22: A non-volatile memory storage device including instructions that, when executed by a processor, perform operations as described in any of clauses 1-20.

Clause 23: A sensor enabled environment (SEE) including a plurality of sensors disposed at various locations that is configured to communicate data from the plurality of sensors related to behavioral, health, wellness, and safety (BHWS) events affecting at least one person under monitoring (PUM) to a linguistic artificial intelligence or machine learning (AI/ML) model configured to monitor the SEE according to a language of wellness (LoW) syntax and to generate alerts in response to identifying that a predicted BHWS event or an observed BHWS disobeys the LOW syntax, wherein the predicted BHWS event is generated based on a series of observed BHWS event for the PUM in the SEE reported to the linguistic AI/ML model.

Although certain figures and descriptions have been provided, many additional variations and modifications will be apparent to those of skill in the art. It will be appreciated that presenting all possible variations and modifications is an impractical task, and thus any sequence, particular structural or device implementation, or underlying technique of the present disclosure may be substituted or modified to meet the needs of particular implementations, and that doing so may not depart from the scope of the present disclosure. It will therefore be appreciated that the examples presented herein are presented for illustrative purposes only, and are not intended to be limiting of a scope of the present disclosure. It will also be apparent to any individual of skill in the art that various embodiments described herein and elements thereof may be combined as needed to suit any particular implementation, and that doing so does not depart from the scope of the present disclosure. As such, the scope of the present disclosure is not to be understood as being limited by the figures or specification presented herein; the scope of the present disclosure should instead be understood in a context of the appended claims and their equivalents.

Certain terms are used throughout the description and claims to refer to particular features or components. As one skilled in the art will appreciate, different persons may refer to the same feature or component by different names. This document does not intend to distinguish between components or features that differ in name but not purpose or operation.

As used herein, the term “optimize” and variations thereof, is used in a sense understood by data scientists to refer to actions taken for continual improvement of a system relative to a goal. An optimized value will be understood to represent “near-best” value for a particular reward framework, which may oscillate around a local maximum or a global maximum for a “best” value or set of values, which may change as the goal changes or as input conditions change. Accordingly, an optimal solution for a first goal at a particular time may be suboptimal for a second goal at that time or suboptimal for the first goal at a later time.

As used herein, “about,” “approximately” and “substantially” are understood to refer to numbers in a range of the referenced number, for example the range of −10% to +10% of the referenced number, preferably-5% to +5% of the referenced number, more preferably-1% to +1% of the referenced number, most preferably-0.1% to +0.1% of the referenced number.

Furthermore, all numerical ranges herein should be understood to include all integers, whole numbers, or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in the present disclosure, the term “or” is to be interpreted in the inclusive sense and not the exclusive sense unless explicitly stated otherwise or when clear from the context. Accordingly, recitation of “A or B” is intended to cover the sets of A, B, and A-B, where the sets may include one or multiple instances of a particular member (e.g., A-A, A-A-A, A-A-B, etc.) and any ordering thereof.

As used in the present disclosure, a phrase referring to “at least one of′ a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, A-C, and A-B-C, where the sets may include one or multiple instances of a particular member (e.g., A-A, A-A-A, A-A-B, A-A-B-B-C-C-C, etc.) and any ordering thereof. For avoidance of doubt, the phrase “at least one of A, B, and C” shall not be interpreted to mean “at least one of A, at least one of B, and at least one of C”.

As used in the present disclosure, the term “determining” encompasses a variety of actions that may include calculating, computing, processing, deriving, investigating, identifying, looking up (e.g., via a table, database, or other data structure), ascertaining, receiving (e.g., receiving information), accessing (e.g., accessing data in a memory), retrieving, resolving, selecting, choosing, establishing, and the like.

Without further elaboration, it is believed that one skilled in the art can use the preceding description to use the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112 (f) unless the element is expressly recited using the phrase “means for” or “step for”. All structural and functional equivalents to the elements of the various embodiments described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

The following listing of claims is intended to replace all prior listings of the claims:

1. A method, comprising:
receiving training data from at least one sensor enabled environment (SEE) related to behavioral, health, wellness, and safety (BHWS) events affecting at least one person under monitoring (PUM), wherein each PUM of the at least one PUM is associated with a corresponding SEE of the at least one SEE;
developing a language of wellness (LoW) syntax for a linguistic artificial intelligence or machine learning (AI/ML) model by aligning the BHWS events with a pattern framework indicative of behaviors of the at least one PUM in the corresponding SEE;
training the linguistic AI/ML model based on occurrences of the BHWS events in the training data and the LoW syntax such that the linguistic AI/ML model is configured to:
generate a predicted BHWS event based on a series of behaviors observed for a particular PUM in a particular SEE reported to the linguistic AI/ML model; and
generate a predictive alert in response to identifying that the predicted BHWS event disobeys the LoW syntax.

2. The method of claim 1, wherein the predicted BHWS event is predictively generated based on one or more digital twins associated with the particular PUM that simulate actions of the particular PUM within the particular SEE.

3. The method of claim 1, wherein linguistic AI/ML model is configured to operate in conjunction with a physics engine associated with the particular PUM to identify when a current BHWS event or the predicted BHWS event is outside of a physical capability of the particular PUM to perform.

4. The method of claim 1, wherein the training data include tokenized representations of various BHWS events, which include encrypted sensor data from the at least one SEE and an unencrypted data label identifying a type of BWHS event associated with the encrypted sensor data.

5. The method of claim 1, wherein the linguistic AI/ML model is one of:
a large language model (LLM);
retrieval augmented generation (RAG) model;
private large language model (PLLM); and
a specialized language model.

6. The method of claim 1, wherein the particular SEE is not included in the at least one SEE from which the training data are received, and the particular PUM is not included in the at least one PUM associated with the at least one SEE.

7. The method of claim 1, wherein the linguistic AI/ML model is further configured to identify an immediate danger state based on a currently or previously observed BHWS event affecting the particular PUM in the particular SEE and to generate an immediate alert based on the immediate danger state.

8. The method of claim 1, wherein disobeying the LoW syntax includes disobeying an established spatial zone in the particular SEE, wherein the predicted BHWS is predicted to occur outside of the established spatial zone.

9. The method of claim 1, wherein disobeying the LoW syntax includes disobeying a time window, wherein the predicted BHWS is predicted to occur outside of the time window, wherein the time window is one of an absolute time window during a day or a relative time window from performance of a previous behavior by the PUM.

10. The method of claim 1, wherein disobeying the LoW syntax includes disobeying an established order for performing a first behavior relative to a second behavior.

11. The method of claim 1, wherein the linguistic AI/ML model is further configured to:
generate a reactive alert in response to identifying that the PUM has performed or is currently performing a behavior that disobeys the LoW syntax.

12. A method, comprising:
deploying a linguistic artificial intelligence or machine learning (AI/ML) model, the linguistic AI/ML model being configured to process prompts according to a language of wellness (LoW) syntax;
receiving a prompt from a computing device located remotely from where the linguistic AI/ML model is deployed, the prompt including at least one tokenized behavioral, health, wellness, and safety (BHWS) event occurring in a particular Sensor Enabled Environment (SEE) associated with the computing device and with a particular person under monitoring (PUM);
generating a predicted BHWS event based on the prompt; and
transmitting the predicted BHWS event to the computing device as an output responsive to the prompt.

13. The method of claim 12, wherein the linguistic AI/ML model is one of:
a large language model (LLM);
retrieval augmented generation (RAG) model;
private large language model (PLLM); and
a specialized language model.

14. The method of claim 12, wherein the particular SEE is not included among training SEE from which training data used to train the linguistic AI/ML model are received, and the particular PUM is not included among training PUM associated with the training SEE.

15. The method of claim 12, wherein the linguistic AI/ML model is further configured to identify an immediate danger state based on a currently or previously observed BHWS event affecting the particular PUM in the particular SEE and to generate an immediate alert based on the immediate danger state.

16. The method of claim 12, further comprising, in response to identifying that the predicted BHWS event disobeys the LoW syntax:
generating a predictive alert; and
transmitting the predictive alert to a stakeholder associated with the particular SEE or the particular PUM.

17. The method of claim 16, wherein disobeying the LoW syntax includes at least one of:
disobeying an established spatial zone in the particular SEE, wherein the predicted BHWS is predicted to occur outside of the established spatial zone;
disobeying the LoW syntax includes disobeying a time window, wherein the predicted BHWS is predicted to occur outside of the time window, wherein the time window is one of an absolute time window during a day or a relative time window from performance of a previous behavior by the PUM; and
disobeying an established order for performing a first behavior relative to a second behavior.

18. The method of claim 16, wherein the linguistic AI/ML model is further configured to:
generate a reactive alert in response to identifying that the PUM has performed or is currently performing a behavior that disobeys the LoW syntax.

19. The method of claim 12, wherein the linguistic AI/ML model is provided for treatment or prophylaxis of a health condition indicated for the particular PUM in a health care plan (HCP) included or referenced in the prompt.

20. The method of claim 12, wherein the prompt includes a senor data stream from at least one sensor disposed in the SEE or a token stream from at least one tokenization service or model that processes sensor data from sensors in the SEE.

21-41. (canceled)
